SEQUENCE LISTING (5 sequences)

SEQ ID NO: 1 - protein, 192 aa, Homo sapiens
  source 1..192: note = Human Rac-1 amino acid sequence NP_008839.2
MQAIKCVVVGDGAVGKTCLLISYTTNAFPGEYIPTVFDNYSANVMVDGKPVNLGLWDTAGQEDYDRLRPLSYPQTDVFLICFSLVSPASFENVRAKWYPEVRHHCPNTPIILVGTKLDLRDDKDTIEKLKEKKLTPITYPQGLAMAKEIGAVKYLECSALTQRGLKTVFDEAIRAVLCPPPVKKRKRKCLLL

SEQ ID NO: 2 - protein, 404 aa, Homo sapiens
  source 1..404: note = Human RAGE amino acid sequence NP_001127.1
MAAGTAVGAWVLVLSLWGAVVGAQNITARIGEPLVLKCKGAPKKPPQRLEWKLNTGRTEAWKVLSPQGGGPWDSVARVLPNGSLFLPAVGIQDEGIFRCQAMNRNGKETKSNYRVRVYQIPGKPEIVDSASELTAGVPNKVGTCVSEGSYPAGTLSWHLDGKPLVPNEKGVSVKEQTRRHPETGLFTLQSELMVTPARGGDPRPTFSCSFSPGLPRHRALRTAPIQPRVWEPVPLEEVQLVVEPEGGAVAPGGTVTLTCEVPAQPSPQIHWMKDGVPLPLPPSPVLILPEIGPQDQGTYSCVATHSSHGPQESRAVSISIIEPGEEGPTAGSVGGSGLGTLALALGILGGLGTAALLIGVILWQRRQRRGEERKAPENQEEEEERAELNQSEEPEAGESSTGGP

SEQ ID NO: 3 - protein, 99 aa, Homo sapiens
  source 1..99: note = Human MCP-1 amino acid sequence NP_002973.1
MKVSAALLCLLLIAATFIPQGLAQPDAINAPVTCCYNFTNRKISVQRLASYRRITSSKCPKEAVIFKTIVAKEICADPKQKWVQDSMDHLDKQTQTPKT

SEQ ID NO: 4 - protein, 360 aa, Homo sapiens
  source 1..360: note = Human CCR2 amino acid sequence NP_001116868.1
MLSTSRSRFIRNTNESGEEVTTFFDYDYGAPCHKFDVKQIGAQLLPPLYSLVFIFGFVGNMLVVLILINCKKLKCLTDIYLLNLAISDLLFLITLPLWAHSAANEWVFGNAMCKLFTGLYHIGYFGGIFFIILLTIDRYLAIVHAVFALKARTVTFGVVTSVITWLVAVFASVPGIIFTKCQKEDSVYVCGPYFPRGWNNFHTIMRNILGLVLPLLIMVICYSGILKTLLRCRNEKKRHRAVRVIFTIMIVYFLFWTPYNIVILLNTFQEFFGLSNCESTSQLDQATQVTETLGMTHCCINPIIYAFVGEKFRRYLSVFFRKHITKRFCKQCPVFYRETVDGVTSTNTPSTGEQEVSAGL

SEQ ID NO: 5 - protein, 374 aa, Homo sapiens
  source 1..374: note = Human CCR2 amino acid sequence NP_001116513.2
MLSTSRSRFIRNTNESGEEVTTFFDYDYGAPCHKFDVKQIGAQLLPPLYSLVFIFGFVGNMLVVLILINCKKLKCLTDIYLLNLAISDLLFLITLPLWAHSAANEWVFGNAMCKLFTGLYHIGYFGGIFFIILLTIDRYLAIVHAVFALKARTVTFGVVTSVITWLVAVFASVPGIIFTKCQKEDSVYVCGPYFPRGWNNFHTIMRNILGLVLPLLIMVICYSGILKTLLRCRNEKKRHRAVRVIFTIMIVYFLFWTPYNIVILLNTFQEFFGLSNCESTSQLDQATQVTETLGMTHCCINPIIYAFVGEKFRSLFHIALGCRIAPLQKPVCGGPGVRPGKNVKVTTQGLLDGRGKGKSIGRAPEASLQDKEGA

(19) United States
(12) Patent Application Publication
(10) Pub. No.: US 2025/0249014 A1
(43) Pub. Date: Aug. 7, 2025

(54) A COMBINATION FOR TREATING A RETINAL DISEASE

(71) Applicants: HADASIT MEDICAL RESEARCH SERVICES AND DEVELOPMENT LTD., Jerusalem (IL); YISSUM RESEARCH DEVELOPMENT COMPANY OF THE HEBREW UNIVERSITY OF JERUSALEM LTD., Jerusalem (IL)

(72) Inventors: Samer KHATEB, Jerusalem (IL); Itay CHOWERS, Jerusalem (IL); Ofra BENNY, Jerusalem (IL)

(73) Assignees: HADASIT MEDICAL RESEARCH SERVICES AND DEVELOPMENT LTD., Jerusalem (IL); YISSUM RESEARCH DEVELOPMENT COMPANY OF THE HEBREW UNIVERSITY OF JERUSALEM LTD., Jerusalem (IL)

(21) Appl. No.: 18/996,086

(22) PCT Filed: Jul. 20, 2023
(86) PCT No.: PCT/IL2023/050760

§ 371 Date: Jan. 17, 2025

Related U.S. Application Data
(60) Provisional application No. 63/368,916, filed on Jul. 20, 2022.

(51) Int. Cl. A61K 31/5377; A61K 31/085; A61K 31/166; A61K 31/167; A61K 31/402; A61K 31/4164; A61K 31/42; A61K 31/454; A61K 31/506; A61P 27/02

(52) U.S. Cl. CPC A61K 31/5377; A61K 31/085; A61K 31/166; A61K 31/167; A61K 31/402; A61K 31/4164; A61K 31/42; A61K 31/454; A61K 31/506; A61P 27/02

(57) ABSTRACT

The present disclosure relates to combination the combination, pharmaceutical compositions and kits comprising at least two small molecule inhibitors (SMIs) of respectively at least two proteins of different signaling pathways of retinal disease and methods for treating a retinal disease.

### TECHNOLOGICAL FIELD

The present disclosure relates to combinations and compositions for treating retinal diseases.

### BACKGROUND ART

References considered to be relevant as background to the presently disclosed subject matter are listed below:
L. P. Aiello, E. A. Pierce, E. D. Foley, H. Takagi, H. Chen, L. Riddle, N. Ferrara, G. L. King, L. E. H. Smith, Suppression of retinal neovascularization in vivo by inhibition of vascular endothelial growth factor (VEGF) using soluble VEGF-receptor chimeric proteins. Proc. Natl. Acad. Sci. U.S.A (1995), doi: 10.1073/pnas.92.23.10457.
M. G. Krzystolik, M. A. Afshari, A. P. Adamis, J. Gaudreault, E. S. Gragoudas, N. A. Michaud, W. Li, E. Connolly, C. A. O'Neill, J. W. Miller, Prevention of experimental choroidal neovascularization with intravitreal anti-vascular endothelial growth factor antibody fragment. Arch. Ophthalmol. (2002), doi: 10.1001/archopht.120.3.338.
Q. D. Nguyen, D. M. Brown, D. M. Marcus, D. S. Boyer, S. Patel, L. Feiner, A. Gibson, J. Sy, A. C. Rundle, J. J. Hopkins, et al Ranibizumab for diabetic macular edema: Results from 2 phase iii randomized trials: RISE and RIDE. Ophthalmology (2012), doi: 10.1016/j.ophtha.2011.12.039.
S. Rofagha, R. B. Bhisitkul, D. S. Boyer, S. R. Sadda, K. Zhang, Seven-year outcomes in ranibizumab-treated patients in ANCHOR, MARINA, and HORIZON: A multicenter cohort study (SEVEN-UP). Ophthalmology (2013), doi: 10.1016/j.ophtha.2013.03.046.
K. G. Falavarjani, Q. D. Nguyen, Adverse events and complications associated with intravitreal injection of anti-VEGF agents: A review of literature. Eye (2013), doi: 10.1038/eye.2013.107.
M. Chen, J. V. Glenn, S. Dasari, C. McVicar, M. Ward, L. Colhoun, M. Quinn, A. Bierhaus, H. Xu, A. W. Stitt, RAGE regulates immune cell infiltration and angiogenesis in choroidal neovascularization. PLOS One (2014), doi: 10.1371/journal.pone.0089548.
H. Wang, M. Elizabeth Hartnett, Regulation of signaling events involved in the pathophysiology of neovascular AMD. Mol. Vis. (2016).
J. Xu, L. J. Chen, J. Yu, H. J. Wang, F. Zhang, Q. Liu, J. Wu, Involvement of Advanced Glycation End Products in the Pathogenesis of Diabetic Retinopathy. Cell. Physiol. Biochem. 48, 705-717 (2018).
N. Sahajpal, A. Kowluru, R. A. Kowluru, The Regulatory Role of Rac1, a Small Molecular Weight GTPase, in the Development of Diabetic Retinopathy. J. Clin. Med. (2019), doi: 10.3390/jcm8070965.
U.S. Pat. No. 8,247,370.
International Patent Application Publication No. WO2018175340.
U.S. Pat. No. 7,279,468.

Acknowledgement of the above references herein is not to be inferred as meaning that these are in any way relevant to the patentability of the presently disclosed subject matter.

### BACKGROUND

Retinal vascular diseases are considered leading causes of blindness and constitute a significant public health issue that calls for an efficient treatment and prevention methods. Particularly, there is a significant worldwide increase in the number of people at risk of reversible and irreversible visual impairments, due to associated pathological angiogenesis of the retina or choroid. Of these diseases, wet (neovascular) age-related macular degeneration (nAMD), diabetic retinopathy (Non-proliferative and proliferative diabetic retinopathy (PDR), diabetic macular oedema (DMO)), and retinal vein occlusion (RVO) are of particular epidemiological importance as leading causes of blindness.

Studies by Aiello et al (1995) and Krzystolik et al (2002) demonstrate casual role of Vascular Endothelial Growth Factor (VEGF) in retinal angiogenesis. Furthermore, these studies show the potential of intravitreal injections of macromolecular VEGF inhibitors as a specific therapy for vascular retinal diseases.

Study by Nguyen et al (2012) demonstrates that Ranibizumab, a monoclonal antibody that inhibits VEGF, rapidly and sustainably improved vision, reduced the risk of further vision loss, and improved macular edema, with low rates of ocular harm.

Study by Rofagha et al (2013) shows that intravitreal injections of anti-VEGF antibodies result in poor outcomes in one third of patients and may cause systemic or ocular complications.

Study by Falavarjani and Nguyen (2013) is a review of literature covering a variety of adverse effects and complications associated with intravitreal injection of anti-VEGF agents.

Study by Wang and Hartnett (2016) describes the essential role of Rac1 in the migration of choroidal EC in the process of creating choroidal neovascularization membrane in nAMD.

Study by Sahajpal et al (2019) describes the role of Ras-related C3 botulinum toxin substrate 1 (Rac1) protein in the pathogenesis of diabetic retinopathy.

Studies by Chen et al (2014) and Xu et al (2018) demonstrate a pivotal role of a receptor for advanced glycation end products (RAGE) in the angiogenesis of wAMD and diabetic retinopathy.

U.S. Pat. No. 8,247,370 describes methods of treating fibrotic or fibroproliferative disorders.

International Patent Application Publication No. WO2018175340 describes polymer conjugates comprising an active agent linked to a polymer, wherein the active agent comprises an inhibitor, antagonist, or inverse agonist of a mediator of a therapeutic target associated with a condition, including for example, an ophthalmic condition, a dermatological condition, an inflammatory bowel disease or other gastrointestinal conditions and a respiratory condition.

U.S. Pat. No. 7,279,468 describes novel compounds which bind to integrin receptors, their use as ligands of integrin receptors and pharmaceutical preparations comprising these compounds.

### GENERAL DESCRIPTION

The present disclosure provides a combination for treating a retinal disease, the combination comprising at least two small molecule inhibitors (SMIs) of respectively at least two proteins of different signaling pathways of retinal disease.

Also provided by the present disclosure is a pharmaceutical composition comprising a pharmaceutically acceptable carrier and at least two small molecule inhibitors (SMIs) of respectively at least two proteins of different signaling pathways associated with at least a retinal disease.

Further provided by the present disclosure is a method of treating a vascular disease, the method comprises administering to a subject in need of said treatment at least two small molecule inhibitors (SMIs) of respectively at least two proteins of different signaling pathways of retinal disease.

### EMBODIMENTS

Some embodiments of this disclosure will now be described in the following numbered paragraph. The following description intends to add on the above general description and not limit it in any manner.
1. A combination comprising at least two inhibitors of respectively at least two proteins of different signaling pathways of retinal disease.
2. The combination of Embodiment No. 1, wherein the at least two proteins are selected from the group consisting of CCR2, MCP-1, RAGE and Rac-1.
3. The combination of Embodiment No. 1, wherein the at least two proteins are selected from the group consisting of CCR2, RAGE and Rac-1.
4. The combination of any one of Embodiments No. 1 to 3, wherein the at least two inhibitors are at least two small molecule inhibitors (SMIs).
5. The combination of Embodiment No. 4, wherein each of the at least two SMIs fulfills one or more of the following criteria:
   it has a molecular weight of less than 600 Da;
   it has no more than 5 hydrogen bond donors;
   it has no more than 10 hydrogen bond acceptors; and
   it has a partition coefficient not greater than 5.
6. The combination of Embodiment No. 4 or 5, wherein the at least two SMIs, at least one SMI inhibits at least the RAGE protein and at least one other SMI inhibits at least the Rac1 protein.
7. The combination of Embodiment No. 4 or 5, wherein the at least two SMIs, at least one SMI inhibits at least the RAGE protein and at least one other SMI inhibits at least the MCP-1 protein.
8. The combination of Embodiment No. 4 or 5, wherein said at least two SMIs, at least one SMI inhibits at least the RAGE protein and at least one other SMI inhibits at least the CCR2 protein.
9. The combination of Embodiment No. 4 or 5, wherein the at least two SMIs, at least one SMI inhibits at least the Rac1 protein and at least one other SMI inhibits at least the MCP-1 protein.
10. The combination of Embodiment No. 4 or 5, wherein the at least two SMIs, at least one SMI inhibits at least the Rac1 protein and at least one other SMI inhibits at least the CCR2 protein.
11. The combination of any one of Embodiments No. 4 to 10, wherein the SMI that inhibits at least the Rac1 protein has a chemical structure of one or more of Formula I, IV, V, VI, VII or a solvate, a hydrate, a stereoisomer, a pharmaceutically acceptable prodrug, a pharmaceutically active metabolite, a pharmaceutically acceptable salt, a crystalline form, an amorphous form, a physiologically functional analogue, a physiologically functional derivative thereof or a combination thereof.
12. The combination of Embodiment No. 11, wherein the SMI has a chemical structure of Formula I.
13. The combination of Embodiment No. 11, wherein the SMI is a functional analog of Formula I.
14. The combination of any one of Embodiments No. 4 to 10, wherein the SMI that inhibits at least the RAGE protein has a chemical structure of one or more of Formula II, VIII, IX or a solvate, a hydrate, a stereoisomer, a pharmaceutically acceptable prodrug, a pharmaceutically active metabolite, a pharmaceutically acceptable salt, a crystalline form, an amorphous form, a physiologically functional analogue, a physiologically functional derivative thereof or a combination thereof.
15. The combination of Embodiment No. 14, wherein the SMI has a chemical structure of Formula II.
16. The combination of Embodiment No. 14, wherein the SMI is a functional analog of Formula II.
17. The combination of any one of Embodiments No. 4 to 10, wherein the SMI that inhibits at least the CCR2 protein has a chemical structure of one or more of Formula III, XI, XII or a solvate, a hydrate, a stereoisomer, a pharmaceutically acceptable prodrug, a pharmaceutically active metabolite, a pharmaceutically acceptable salt, a crystalline form, an amorphous form, a physiologically functional analogue, a physiologically functional derivative thereof or a combination thereof.
18. The combination of Embodiment No. 17, wherein the SMI has a chemical structure of Formula III.
19. The combination of Embodiment No. 17, wherein the SMI is a functional analog of Formula III.
20. The combination of any one of Embodiments No. 4 to 19, wherein the at least two SMIs are selected from (i) at least one SMI having Formula (I), (IV), (V), (VI) or (VII); (ii) at least one SMI having Formula (II), (VIII), (IX) or (X); (iii) at least one SMI having Formula (III), (XI) or (XII) or a solvate, a hydrate, a stereoisomer, a pharmaceutically acceptable prodrug, a pharmaceutically active metabolite, a pharmaceutically acceptable salt, a crystalline form, an amorphous form, a physiologically functional analogue, a physiologically functional derivative thereof or a combination thereof.
21. The combination of any one of Embodiments No. 4 to 20, wherein the at least two SMIs are selected from (i) at least one SMI having Formula (I), (IV) or (V); (ii) at least one SMI having Formula (II), (VIII), (IX) or (X); (iii) at least one SMI having Formula (III), (XI) or (XII) or a solvate, a hydrate, a stereoisomer, a pharmaceutically acceptable prodrug, a pharmaceutically active metabolite, a pharmaceutically acceptable salt, a crystalline form, an amorphous form, a physiologically functional analogue, a physiologically functional derivative thereof or a combination thereof.
22. The combination of any one of Embodiments No. 1 to 21, being a synergistic combination.
23. The combination of Embodiment No. 22, wherein the at least two SMIs are each in a synergistic amount.
24. The combination of any one of Embodiments No. 1 to 23, for use in treating a retinal disease.
25. The combination of Embodiment No. 24, wherein the retinal disease is selected from the group consisting of wet (neovascular) age-related macular degeneration (nAMD), diabetic retinopathy (DR, including proliferative diabetic retinopathy (PDR), and non-proliferative diabetic retinopathy (nPDR)), diabetic macular oedema (DMO), retinopathy of prematurity, radiation retinopathy, hypertensive retinopathy, myopic choroidal neovascularization (CNV), retinal vein occlusion (RVO), retinal artery occlusion (RAO), vasoproliferative tumor, coat's diseases, familial exudative vitreoretinopathy (FEVR), uveitis induced CNV, inherited retinal degeneration associated CNV, sickle cell retinopathy, idiopathic choroidal neovascularization, Irvine-Gass syndrome, choroidal hemangioma, dry age-related macular degeneration (AMD), retinal atrophy, carcinoma associated retinopathy (CAR), autoimmune induced retinopathy (AIR), central serous chorioretinopathy (CSCR), uveitis, inherited retinal degeneration (IRD), cystoid macular edema, proliferative vitreoretinopathy (PVR), Polypoidal choroidal vasculopathy (PCV).
26. The combination of Embodiment No. 24 or 25, wherein the retinal disease is a retinal vascular disease.
27. The combination of Embodiment No. 26, wherein the retinal vascular disease is selected from the group consisting of wet (neovascular) age-related macular degeneration (nAMD), diabetic retinopathy (DR), proliferative diabetic retinopathy (PDR) and diabetic macular oedema (DMO), and retinal vein occlusion (RVO).
28. The combination of any one of Embodiments No. 1 to 27, wherein at least one of the at least two inhibitors is formulated within a delivery system.
29. The combination of Embodiment No. 28, wherein the delivery system is for controlled delivery of the SMI.
30. The combination of any one of Embodiments No. 1 to 27, wherein the at least two inhibitors are formulated for sequential or concomitant administration.
31. The combination of any one of Embodiments No. 1 to 27, wherein the at least two inhibitors are formulated in the same or different composition.
32. A pharmaceutical composition comprising a pharmaceutically acceptable carrier and at least two inhibitors of respectively at least two proteins of different signaling pathways of retinal disease.
33. The pharmaceutical composition of Embodiment No. 32, wherein the at least two proteins are selected from the group consisting of CCR2, RAGE and Rac-1.
34. The pharmaceutical composition of Embodiment No. 32, wherein the at least two proteins are selected from the group consisting of MCP-1, RAGE and Rac-1.
35. The pharmaceutical composition of any one of the preceding Embodiments, wherein the at least two inhibitors are at least two small molecule inhibitors (SMIs).
36. The pharmaceutical composition of any one of the preceding Embodiments, wherein each of the at least two SMIs fulfills one or more of the following criteria:
   it has a molecular weight of less than 600 Da;
   it has no more than 5 hydrogen bond donors;
   it has no more than 10 hydrogen bond acceptors; and
   it has a partition coefficient not greater than 5.
37. The pharmaceutical composition of any one of the preceding Embodiments, wherein the at least two SMIs, at least one SMI inhibits at least the RAGE protein and at least one other SMI inhibits at least the Rac1 protein.
38. The pharmaceutical composition of any one of the preceding Embodiments, wherein the at least two SMIs, at least one SMI inhibits at least the RAGE protein and at least one other SMI inhibits at least the MCP-1 protein.
39. The pharmaceutical composition of any one of the preceding Embodiments, wherein the at least two SMIs, at least one SMI inhibits at least the RAGE protein and at least one other SMI inhibits at least the CCR2 protein.
40. The pharmaceutical composition of any one of the preceding Embodiments, wherein the at least two SMIs, at least one SMI inhibits at least the Rac1 protein and at least one other SMI inhibits at least the MCP-1 protein.
41. The pharmaceutical composition of any one of the preceding Embodiments, wherein the at least two SMIs, at least one SMI inhibits at least the Rac1 protein and at least one other SMI inhibits at least the CCR2 protein.
42. The pharmaceutical composition of any one of the preceding Embodiments, wherein the SMI that inhibits at least the Rac1 protein has a chemical structure of one or more of Formula I, IV, V, VI, VII or a solvate, a hydrate, a stereoisomer, a pharmaceutically acceptable prodrug, a pharmaceutically active metabolite, a pharmaceutically acceptable salt, a crystalline form, an amorphous form, a physiologically functional analogue, a physiologically functional derivative thereof or a combination thereof.
43. The pharmaceutical composition of any one of the preceding Embodiments, wherein the SMI has a chemical structure of Formula I.
44. The pharmaceutical composition of any one of the preceding Embodiments, wherein the SMI is a functional analog of Formula I.
45. The pharmaceutical composition of any one of the preceding Embodiments, wherein the SMI that inhibits at least the RAGE protein has a chemical structure of one or more of Formula II, VIII, IX or a solvate, a hydrate, a stereoisomer, a pharmaceutically acceptable prodrug, a pharmaceutically active metabolite, a pharmaceutically acceptable salt, a crystalline form, an amorphous form, a physiologically functional analogue, a physiologically functional derivative thereof or a combination thereof.
46. The pharmaceutical composition of any one of the preceding Embodiments, wherein the SMI has a chemical structure of Formula II.
47. The pharmaceutical composition of any one of the preceding Embodiments, wherein the SMI is a functional analog of Formula II.
48. The pharmaceutical composition of any one of the preceding Embodiments, wherein the SMI that inhibits at least the CCR2 protein has a chemical structure of one or more of Formula III, XI, XII or a solvate, a hydrate, a stereoisomer, a pharmaceutically acceptable prodrug, a pharmaceutically active metabolite, a pharmaceutically acceptable salt, a crystalline form, an amorphous form, a physiologically functional analogue, a physiologically functional derivative thereof or a combination thereof.
49. The pharmaceutical composition of any one of the preceding Embodiments, wherein the SMI has a chemical structure of Formula III.
50. The pharmaceutical composition of any one of the preceding Embodiments, wherein the SMI is a functional analog of Formula III.
51. The pharmaceutical composition of any one of the preceding Embodiments, wherein the at least two SMIs are selected from (i) at least one SMI having Formula (I), (IV), (V), (VI) or (VII); (ii) at least one SMI having Formula (II), (VIII), (IX) or (X); (iii) at least one SMI having Formula (III), (XI) or (XII) or a solvate, a hydrate, a stereoisomer, a pharmaceutically acceptable prodrug, a pharmaceutically active metabolite, a pharmaceutically acceptable salt, a crystalline form, an amorphous form, a physiologically functional analogue, a physiologically functional derivative thereof or a combination thereof.
52. The pharmaceutical composition of any one of the preceding Embodiments, wherein the at least two SMIs are selected from (i) at least one SMI having Formula (I), (IV) or (V); (ii) at least one SMI having Formula (II), (VIII), (IX) or (X); (iii) at least one SMI having Formula (III), (XI) or (XII) or a solvate, a hydrate, a stereoisomer, a pharmaceutically acceptable prodrug, a pharmaceutically active metabolite, a pharmaceutically acceptable salt, a crystalline form, an amorphous form, a physiologically functional analogue, a physiologically functional derivative thereof or a combination thereof.
53. The pharmaceutical composition of any one of the preceding Embodiments for use in the treatment of a retinal disease.
54. The pharmaceutical composition for use of any one of the preceding Embodiments, wherein the retinal disease is selected from the group consisting of nAMD, DR, PDR, nPDR, DMO, retinopathy of prematurity, radiation retinopathy, hypertensive retinopathy, myopic CNV, RVO, RAO), vasoproliferative tumor, coat's diseases, FEVR, uveitis induced CNV, inherited retinal degeneration associated CNV, sickle cell retinopathy, idiopathic choroidal neovascularization, Irvine-Gass syndrome, choroidal hemangioma, dry AMD, retinal atrophy, CAR, AIR, CSCR, uveitis, IRD, cystoid macular edema, PVR, PCV.
55. The pharmaceutical composition for use of any one of the preceding Embodiments, wherein the retinal disease is a retinal vascular disease.
56. The pharmaceutical composition for use of any one of the preceding Embodiments, wherein the retinal vascular disease is selected from the group consisting of nAMD, DR, PDR, DMO, and RVO.
57. The pharmaceutical composition for use of any one of the preceding Embodiments, formulated for topical administration.
58. The pharmaceutical composition for use of any one of the preceding Embodiments, wherein at least one of the at least two SMIs is formulated within a delivery system.
59. The pharmaceutical composition for use of any one of the preceding Embodiments, wherein the delivery system is for controlled delivery of the SMI.
60. The pharmaceutical composition for use of any one of the preceding Embodiments, wherein the pharmaceutically acceptable carrier is selected from the group comprising polylactic acid (PLA), poly-lactic-co-glycolic acid (PLGA), polyvinyl alcohol (PVA), polyethyleneimine (PEI) and combinations thereof.
61. The pharmaceutical composition for use of any one of the preceding Embodiments, being a synergistic composition.
62. The pharmaceutical composition for use of any one of the preceding Embodiments, wherein the at least two inhibitors are each in a synergistic amount.
63. A method of treating a retinal disease, the method comprises administering to a subject in need of the treatment at least two inhibitors of respectively at least two proteins of different signaling pathways of retinal disease.
64. The method of Embodiment No. 63, wherein the at least two proteins are selected from the group consisting of CCR2, MCP-1, RAGE and Rac-1.
65. The method of Embodiment No. 63, wherein the at least two proteins are selected from the group consisting of CCR2, RAGE and Rac-1.
66. The method of any one of the preceding Embodiments, wherein the at least two inhibitors are at least two small molecule inhibitors (SMIs).
67. The method of any one of the preceding Embodiments, wherein each of the at least two SMIs fulfills one or more of the following criteria:
   it has a molecular weight of less than 600 Da;
   it has no more than 5 hydrogen bond donors;
   it has no more than 10 hydrogen bond acceptors; and
   it has a partition coefficient not greater than 5.
68. The method of any one of the preceding Embodiments, wherein the at least two SMIs, at least one SMI inhibits at least the RAGE protein and at least one other SMI inhibits at least the Rac1 protein.
69. The method of any one of the preceding Embodiments, wherein the at least two SMIs, at least one SMI inhibits at least the RAGE protein and at least one other SMI inhibits at least the MCP-1 protein.
70. The method of any one of the preceding Embodiments, wherein the at least two SMIs, at least one SMI inhibits at least the RAGE protein and at least one other SMI inhibits at least the CCR2 protein.
71. The method of any one of the preceding Embodiments, wherein the at least two SMIs, at least one SMI inhibits at least the Rac1 protein and at least one other SMI inhibits at least the MCP-1 protein.
72. The method of any one of the preceding Embodiments, wherein the at least two SMIs, at least one SMI inhibits at least the Rac1 protein and at least one other SMI inhibits at least the CCR2 protein.
73. The method of any one of the preceding Embodiments, wherein the SMI that inhibits at least the Rac1 protein has a chemical structure of one or more of Formula I, IV, V, VI, VII or a solvate, a hydrate, a stereoisomer, a pharmaceutically acceptable prodrug, a pharmaceutically active metabolite, a pharmaceutically acceptable salt, a crystalline form, an amorphous form, a physiologically functional analogue, a physiologically functional derivative thereof or a combination thereof.
74. The method of any one of the preceding Embodiments, wherein the SMI has a chemical structure of Formula I.
75. The method of any one of the preceding Embodiments, wherein the SMI is a functional analog of Formula I.
76. The method of any one of the preceding Embodiments, wherein the SMI that inhibits at least the RAGE protein has a chemical structure of one or more of Formula II, VIII, IX or a solvate, a hydrate, a stereoisomer, a pharmaceutically acceptable prodrug, a pharmaceutically active metabolite, a pharmaceutically acceptable salt, a crystalline form, an amorphous form, a physiologically functional analogue, a physiologically functional derivative thereof or a combination thereof.
77. The method of any one of the preceding Embodiments, wherein the SMI has a chemical structure of Formula II.
78. The method of any one of the preceding Embodiments, wherein the SMI is a functional analog of Formula II.
79. The method of any one of the preceding Embodiments, wherein the SMI that inhibits at least the CCR2 protein has a chemical structure of one or more of Formula III, XI, XII or a solvate, a hydrate, a stereoisomer, a pharmaceutically acceptable prodrug, a pharmaceutically active metabolite, a pharmaceutically acceptable salt, a crystalline form, an amorphous form, a physiologically functional analogue, a physiologically functional derivative thereof or a combination thereof.
80. The method of any one of the preceding Embodiments, wherein the SMI has a chemical structure of Formula III.
81. The method of any one of the preceding Embodiments, wherein the SMI is a functional analog of Formula III.
82. The method of any one of the preceding Embodiments, wherein the at least two SMIs are selected from (i) at least one SMI having Formula (I), (IV), (V), (VI) or (VII); (ii) at least one SMI having Formula (II), (VIII), (IX) or (X); (iii) at least one SMI having Formula (III), (XI) or (XII) or a solvate, a hydrate, a stereoisomer, a pharmaceutically acceptable prodrug, a pharmaceutically active metabolite, a pharmaceutically acceptable salt, a crystalline form, an amorphous form, a physiologically functional analogue, a physiologically functional derivative thereof or a combination thereof.
83. The method of any one of the preceding Embodiments, wherein the at least two SMIs are selected from (i) at least one SMI having Formula (I), (IV) or (V); (ii) at least one SMI having Formula (II), (VIII), (IX) or (X); (iii) at least one SMI having Formula (III), (XI) or (XII) or a solvate, a hydrate, a stereoisomer, a pharmaceutically acceptable prodrug, a pharmaceutically active metabolite, a pharmaceutically acceptable salt, a crystalline form, an amorphous form, a physiologically functional analogue, a physiologically functional derivative thereof or a combination thereof.
84. The method of any one of the preceding Embodiments, wherein the retinal disease is selected from the group consisting of nAMD, DR, PDR, nPDR, DMO, retinopathy of prematurity, radiation retinopathy, hypertensive retinopathy, myopic CNV, RVO, RAO), vasoproliferative tumor, coat's diseases, FEVR, uveitis induced CNV, inherited retinal degeneration associated CNV, sickle cell retinopathy, idiopathic choroidal neovascularization, Irvine-Gass syndrome, choroidal hemangioma, dry AMD, retinal atrophy, CAR, AIR, CSCR, uveitis, IRD, cystoid macular edema, PVR, PCV.
85. The method of any one of the preceding Embodiments, wherein the retinal disease is a retinal vascular disease.
86. The method of any one of the preceding Embodiments, wherein the retinal vascular disease is selected from the group consisting of nAMD, DR, PDR, DMO, and RVO.
87. The method of any one of the preceding Embodiments, comprising administering at least one of the at least two SMIs topically.
88. The method of any one of the preceding Embodiments, comprising administering at least one of the at least two SMIs by injection.
89. The method of any one of the preceding Embodiments, wherein at least one of the at least two SMIs is formulated within a delivery system.
90. The method of any one of the preceding Embodiments, wherein the delivery system is for controlled delivery of the SMI.
91. The method of any one of the preceding Embodiments, comprising sequential or concomitant administration of the at least two SMIs.
92. The method of any one of the preceding Embodiments, comprising administration of the at least two SMIs in the same composition.
93. A package or kit comprising
   a first SMI targeted against a protein of a signaling pathway of a retinal disease;
   at least one additional SMI being different from the first SMI and targeted at a different protein of a signaling pathway of the retinal disease; and
   instructions for use of a combination of the first SMI and the at least one additional SMI for treating the retinal disease.
94. The package or kit of Embodiment No. 93, wherein the first SMI is separated from the at least one additional SMI.
95. The package or kit of Embodiment No. 93 or 94, comprising a combination and/or a pharmaceutical composition of any one of the preceding Embodiments.
96. Use of at least two SMIs of respectively at least two proteins of different signaling pathways of retinal disease in the preparation of a combination or a pharmaceutical composition for treating a retinal disease.
97. The use of Embodiment No. 96, wherein the combination and/or the pharmaceutical composition of any one of the preceding Embodiments.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIG. 1 is a flow chart showing the WST-1 proliferation screening test according to some examples of the present disclosure.

FIGS. 2A-2B are histograms showing dose-proliferation inhibition relationships for EHop-016 on Human Umbilical Vein Endothelial Cells (HUVECs) at cell density of 3×10³ cells/well (FIG. 2A) and of 1×10³ cells/well (FIG. 2B) according to some examples of the present disclosure.

FIGS. 3A-3C are histograms showing dose-proliferation inhibition relationships for RAGE protein inhibitors on HUVECs according to some examples disclosed herein, specifically for 4′-Methoxyresveratrol (FIG. 3A), FPS-ZM1 (FIG. 3B) and Azeliragon (FIG. 3C).

FIG. 4 is a histogram showing the effect of daily dosing of inhibitors, Azeliragon, EHop-016 and CCR2-RA [R] on HUVECs proliferation according to some examples of the present disclosure.

FIG. 5 is a histogram showing the results of proliferation inhibition of selected combinations of SMIs versus treatment time according to some examples of the present disclosure.

FIGS. 6A-6C are graphs showing synergistic interactions between Azeliragon and EHop-016 on HUVECs proliferation, ZIP model (FIG. 6A), Loewe model (FIG. 6B) and HSA model (FIG. 6C).

FIGS. 7A and 7B show effect of combinations of SMIs according to some examples f the present disclosure on hypoxia-inducible factor (Hif)-1α expression, FIG. 7A is a western blot; 1—control, 2—Azeliragon, 3—EHop-016, 4—Azeliragon and EHop-016, FIG. 7B is a histogram showing analysis of data presented in FIG. 7A using actin as a housekeeping gene for protein quantification.

FIGS. 8A-8C are micrographs taken 11 hours post treatment showing the influence of Rac1 inhibitor EHop-016 on migration of HUVECs according to some examples cited herein, and specifically for untreated cells (FIG. 8A), cells treated with DMSO (FIG. 8B) and cells treated with 0.8 μM of EHop-016 (FIG. 8C).

FIGS. 9A-9C are micrographs taken 13 hours post treatment showing the influence of CCR2-RA-[R] on migration of HUVECs according to some examples cited herein, and specifically for untreated cells (FIG. 9A), cells treated with DMSO (FIG. 9B), and cells treated with 5 μM of CCR2-RA-[R] (FIG. 9C).

FIG. 10 is a histogram showing the effect of SMIs on proliferation of HUVECs, HEK-293 and fibroblast cells according to some examples of the present disclosure.

FIGS. 11A-11F are micrographs (FIGS. 11A-11E) showing inhibition of microangiogenesis in choroid sprouting model following different SMIs treatments according to some examples disclosed herein and specifically for control without DMSO solvent (FIG. 11A), control with DMSO solvent (FIG. 11B), 0.55 μM of RAGE inhibitor Azeliragon (FIG. 11C), 0.70 M of Rac1 inhibitor EHop-016 (FIG. 11D), and a combination of 0.55 μM of RAGE inhibitor Azeliragon combined with 0.70 μM of Rac1 inhibitor EHop-016 (FIG. 11E); and a histogram (FIG. 11F) showing the effect of RAGE, Rac1 and a combined treatment with SMIs on microangiogenesis of choroid explants according to some examples of the present disclosure.

FIGS. 12A and 12B are micrographs showing isolectin staining in sham mice (FIG. 12A) and mice treated with a combination of SMIs according to some examples cited herein (FIG. 12B).

FIG. 13 is a graph showing CNV area in sham mice and mice treated with combinations of SMIs according to some examples cited herein.

FIG. 14 is a graph showing CNV area in sham mice and mice treated with SMIs according to some examples cited herein and specifically sham, Azeliragon (15 μM), EHop-016 (30 μM) and Aflibercept (Eylea®) (41.27 μM) according to some examples of the present disclosure.

### DETAILED DESCRIPTION OF EMBODIMENTS

The present disclosure is based on the finding that treatment of ocular diseases benefits from use of combination of inhibitors such as Small Molecule Inhibitors (SMIs), each pointed at modeling different key target pathways.

Specifically, the present disclosure is based on the finding that combinations of inhibitors, such as SMI combinations, targeted at different signaling pathways related to retinal diseases, provided an unexpected beneficiary advantage in treatment a tested disease as compared to the effect obtained by each inhibitor when given alone in the same test model.

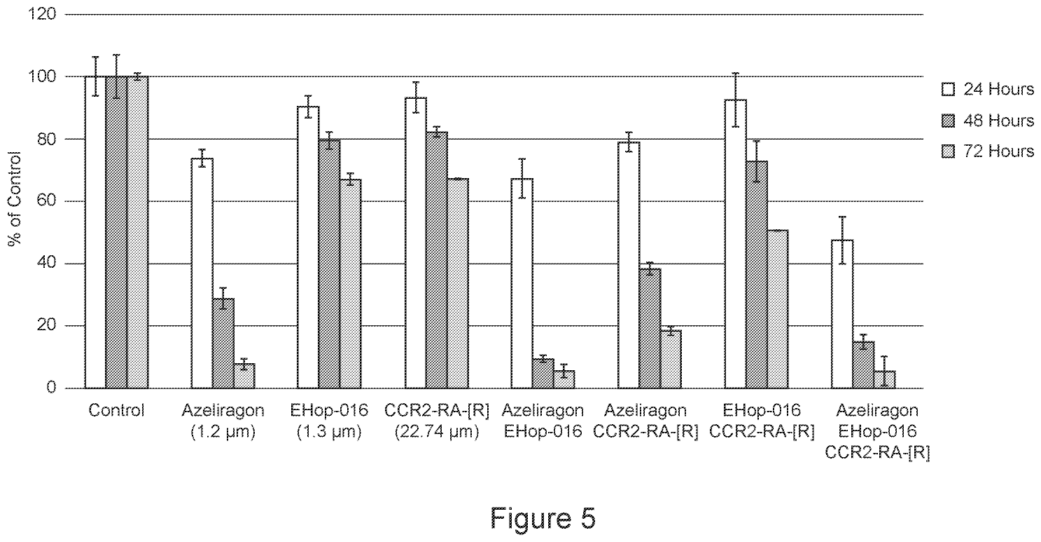

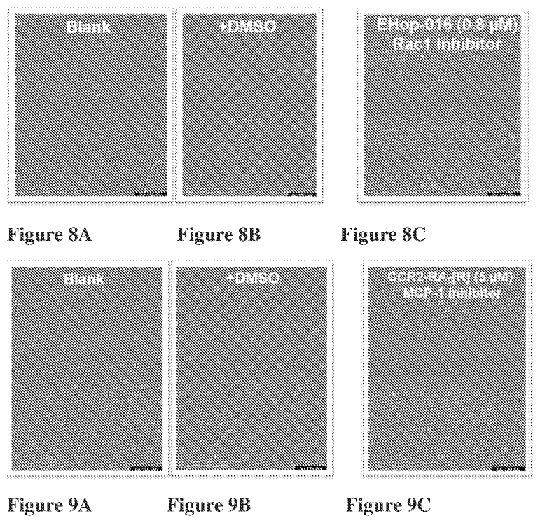

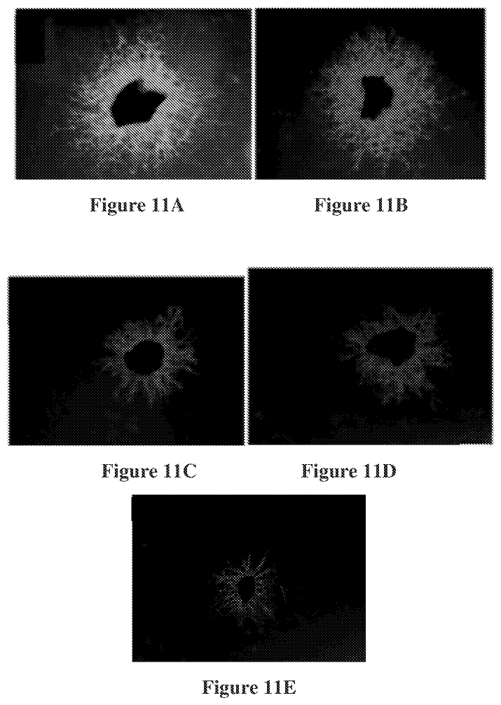

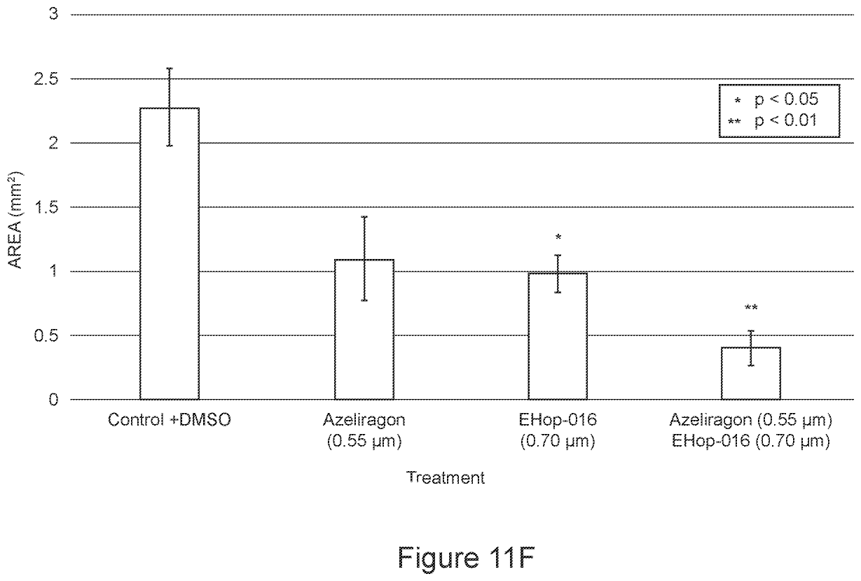

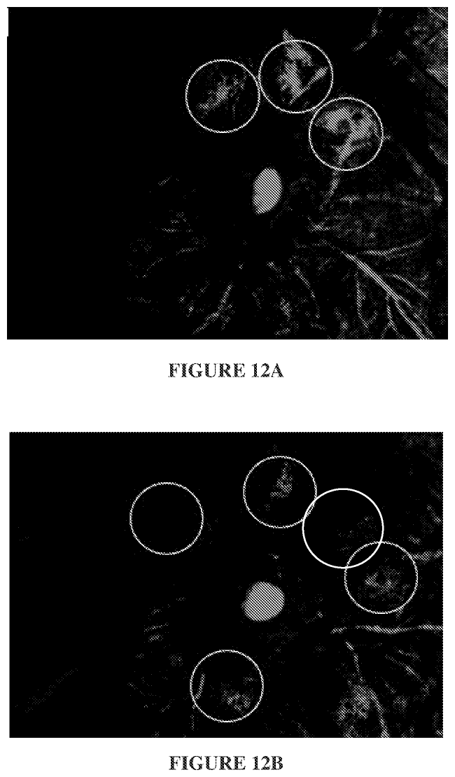

As shown in the Examples below, each one of the different pathways was associated with a different protein such that the overall activity resulted in inhibition of angiogenesis. Specifically, as shown herein, combinations of two or more inhibitors inhibited human umbilical vein endothelial cells (HUVEC) proliferation (FIG. 5), HUVEC migration (FIGS. 8A-8C and 9A-9C), and endothelial cell sprouting (FIGS. 11A-11F).

Based on the results, it was suggested that inhibiting the activity of these proteins using two or more inhibitors, would be useful in inhibiting angiogenesis in the retina and hence in inhibiting retinal damage and treating various retinal diseases. Angiogenesis as used herein refers to a process involving coordinated steps of endothelial cells, including endothelial cells proliferation, endothelial cells migration and capillary sprouting.

Hence, in its broadest aspect, the present disclosure provides a combination comprising at least two inhibitors of respectively at least two proteins of different signaling pathways for use in one or more of inhibiting endothelial cells proliferation, inhibiting endothelial cells migration, inhibiting tube formation, inhibiting capillary sprouting inhibiting retinal damage, treating retinal diseases or any combination thereof.

In the following text, when referring to the combination it is to be understood as also referring to the two or more inhibitors, two or more SMIs, pharmaceutical compositions, methods, uses, and kits disclosed herein. Thus, whenever providing a feature with reference to the combination, it is to be understood as defining the same feature with respect to the two or more inhibitors, two or more SMIs, pharmaceutical compositions, methods, uses, and kit, mutatis mutandis.

As described herein, the disclosed combination was found to be effective at different processes involved in angiogenesis and thus are considered to be useful for various retinal diseases.

In the context of the present disclosure, when referring to a “retinal disease” it is to be understood to encompass any abnormal condition primarily affecting the normal integrity and/or functionality of retina or choroid.

The inhibitors, for examples SMIs of the combination, are inhibitors of different proteins along the signaling pathway of the disease to be treated thereby. There are different signaling pathways associated with retinal diseases and thus different targets for the SMI of the disclosed combination.

In some examples, the inhibitor, for examples SMI is directed against two proteins of different signaling pathways associated with/involved with at least one retinal disease. The signaling pathway associated with a retinal disease can be independently selected from oxidative stress-related pathway, e.g. oxidative-angiogenesis-inflammation pathway; inflammation-related pathway, e.g. inflammation-angiogenesis pathway; PKC activation pathway; Advanced Glycation End Products (AGE) pathway; VEGF-mediated pathways, polyol pathway; retinal ischemic pathway, Toll-Like receptor signaling pathway; VEGF-independent angiogenesis; immune system-related pathways; ischemic pathways. unfolded protein response pathway, mammalian target of rapamycin (mTOR)/Akt pathways, inflammatory cytokine-mediated pathways including tumor necrosis factor alpha (TNF-α) mediated pathways, hepatocyte growth factor (HGF)/c-Met receptor-mediated pathways, Wnt/LRP6-mediated pathway, platelet-derived growth factor (PDGF)/PDGFR-mediated pathway, transforming growth factor β (TGF-β)/TGF-β receptor-mediated pathway, fibroblast growth factors (FGFs)-mediated pathway, TNF-α/TNFR-mediated pathway, and eotaxin/CCR3-mediated pathway, polyol pathway, protein kinase C (PKC) pathway, advanced glycation end products (AGEs) pathway, renin-angiotensin pathway, mitogen activated protein kinase (MAP kinase) pathway, the angiogenic pathway, and pathways associated with oxidative damage mediated by any one of NF-kB, AP-1, p53, HIF-1a, PPAR-γ, and β-catenin/Wnt, pathways mediated by proinflammatory factors (interleukin-6, interleukin-8, monocyte chemoattractant protein-1 (MCP-1) and intercellular adhesion molecule-1), oxidative product-mediated pathways (e.g., NO-mediated pathway).

In some examples, the combination comprising at least two inhibitors of respectively at least two proteins for use in one or more of inhibiting endothelial cells proliferation, inhibiting endothelial cells migration, inhibiting capillary sprouting inhibiting retinal damage, treating retinal diseases or any combination thereof, wherein the at least two proteins are of different signaling pathways associated with a retinal disease and comprising one or more of oxidative stress-related pathway, e.g. oxidative-angiogenesis-inflammation pathway; inflammation-related pathway, e.g. inflammation-angiogenesis pathway; PKC activation pathway; Advanced Glycation End Products (AGE) pathway; VEGF-mediated pathways, polyol pathway; retinal ischemic pathway, Toll-Like receptor signaling pathway; VEGF-independent angiogenesis; immune system-related pathways; ischemic pathways. unfolded protein response pathway, mammalian target of rapamycin (mTOR)/Akt pathways, inflammatory cytokine-mediated pathways including tumor necrosis factor alpha (TNF-α) mediated pathways, hepatocyte growth factor (HGF)/c-Met receptor-mediated pathways, Wnt/LRP6-mediated pathway, platelet-derived growth factor (PDGF)/PDGFR-mediated pathway, transforming growth factor β (TGF-β)/TGF-β receptor-mediated pathway, fibroblast growth factors (FGFs)-mediated pathway, TNF-α/TNFR-mediated pathway, and eotaxin/CCR3-mediated pathway, polyol pathway, protein kinase C (PKC) pathway, advanced glycation end products (AGEs) pathway, renin-angiotensin pathway, mitogen activated protein kinase (MAP kinase) pathway, the angiogenic pathway, and pathways associated with oxidative damage mediated by any one of NF-kB, AP-1, p53, HIF-1α, PPAR-γ, and β-catenin/Wnt, pathways mediated by proinflammatory factors (interleukin-6, interleukin-8, monocyte chemoattractant protein-1 (MCP-1) and intercellular adhesion molecule-1), oxidative product-mediated pathways (e.g., NO-mediated pathway) or any combination thereof.

In some examples, the inhibitor, for example, the SMI is not an inhibitor of vascular endothelial growth factor (VEGF), i.e., does not directly bind and inhibit the activity of VEGF.

In some examples, the at least two proteins against which the two different inhibitors act are selected from the group consisting of a GTPases, immunoglobulin superfamily and a chemokine receptor (CCR).

In some examples, the at least two proteins against which the two different inhibitors act are selected from the group consisting of Ras-related C3 botulinum toxin substrate 1 (Rac1), Receptor for Advanced Glycation End product (RAGE) and chemokine receptor type 2 (CCR2).

Rac1 protein as used herein refers to a small signaling G protein of the Rac subfamily of the Rho family of GTPases.

In some examples, Rac1 protein is human Rac1.

In some examples, Rac1 protein may comprise the amino acid sequence as denoted by NCBI Reference Sequence: NP_008839.2.

In some embodiments, the amino acid sequence of Rac1 is as denoted by SEQ ID NO: 1. SEQ ID NO:1 is provided by the following amino acid sequence (denoted by the single letter code)

MQAIKCVVVGDGAVGKTCLLISYTTNAFPGEYIPTVFDNYSANVMVDGK
PVNLGLWDTAGQEDYDRLRPLSYPQTDVFLICFSLVSPASFENVRAKWY
PEVRHHCPNTPIILVGTKLDLRDDKDTIEKLKEKKLTPITYPQGLAMAK
EIGAVKYLECSALTQRGLKTVFDEAIRAVLCPPPVKKRKRKCLLL

RAGE protein (also called AGER) as used herein refers to a transmembrane receptor of the immunoglobulin superfamily and has the ability to bind Advanced Glycation End products (AGE). RAGE exists in the body in two forms: a membrane-bound form known as mRAGE, and a soluble form, known as sRAGE.

The full receptor consists of a cytosolic domain (responsible for signal transduction), transmembrane domain (anchors the receptor in the cell membrane), a variable domain (binds the RAGE ligands, and two constant domains).

In some examples, RAGE protein is human RAGE.

In some examples, RAGE protein may comprise the amino acid sequence as denoted by NCBI Reference Sequence: NP_001127.1.

In some examples, the amino acid sequence of RAGE is as denoted by SEQ ID NO: 2. SEQ ID NO:2 is provided by the following amino acid sequence (denoted by the single letter code)

MAAGTAVGAWVLVLSLWGAVVGAQNITARIGEPLVLKCKGAPKKPPQRL
EWKLNTGRTEAWKVLSPQGGGPWDSVARVLPNGSLFLPAVGIQDEGIFR
CQAMNRNGKETKSNYRVRVYQIPGKPEIVDSASELTAGVPNKVGTCVSE
GSYPAGTLSWHLDGKPLVPNEKGVSVKEQTRRHPETGLFTLQSELMVTP
ARGGDPRPTFSCSFSPGLPRHRALRTAPIQPRVWEPVPLEEVQLVVEPE
GGAVAPGGTVTLTCEVPAQPSPQIHWMKDGVPLPLPPSPVLILPEIGPQ
DQGTYSCVATHSSHGPQESRAVSISIIEPGEEGPTAGSVGGSGLGTLAL
ALGILGGLGTAALLIGVILWQRRQRRGEERKAPENQEEEEERAELNQSE
EPEAGESSTGGP

CCR2 (also known as CD192) as used herein refers to a member of the CC chemokine receptor family of G protein-linked receptors that are known as seven transmembrane (7-TM) proteins since they span the cell membrane seven times.

In some examples, the CCR2 protein is human CCR2.

In some examples, CCR2 protein may comprise the amino acid sequence as denoted by NCBI Reference Sequence: NP_001116868.1.

In more examples, the amino acid sequence of CCR2 is as denoted by SEQ ID NO: 4. SEQ ID NO:4 is provided by the following amino acid sequence (denoted by the single letter code)

MLSTSRSRFIRNTNESGEEVTTFFDYDYGAPCHKFDVKQIGAQLLPPLY
SLVFIFGFVGNMLVVLILINCKKLKCLTDIYLLNLAISDLLFLITLPLW
AHSAANEWVFGNAMCKLFTGLYHIGYFGGIFFIILLTIDRYLAIVHAVF
ALKARTVTFGVVTSVITWLVAVFASVPGIIFTKCQKEDSVYVCGPYFPR
GWNNFHTIMRNILGLVLPLLIMVICYSGILKTLLRCRNEKKRHRAVRVI
FTIMIVYFLFWTPYNIVILLNTFQEFFGLSNCESTSQLDQATQVTETLG
MTHCCINPIIYAFVGEKFRRYLSVFFRKHITKRFCKQCPVFYRETVDGV
TSTNTPSTGEQEVSAGL

In some examples, CCR2 protein may comprise the amino acid sequence as denoted by NCBI Reference Sequence: NP_001116513.2.

In more specific examples, the amino acid sequence of CCR2 is as denoted by SEQ ID NO: 5. SEQ ID NO:5 is provided by the following amino acid sequence (denoted by the single letter code)

MLSTSRSRFIRNTNESGEEVTTFFDYDYGAPCHKFDVKQIGAQLLPPLY
SLVFIFGFVGNMLVVLILINCKKLKCLTDIYLLNLAISDLLFLITLPLW
AHSAANEWVFGNAMCKLFTGLYHIGYFGGIFFIILLTIDRYLAIVHAVF
ALKARTVTFGVVTSVITWLVAVFASVPGIIFTKCQKEDSVYVCGPYFPR
GWNNFHTIMRNILGLVLPLLIMVICYSGILKTLLRCRNEKKRHRAVRVI
FTIMIVYFLFWTPYNIVILLNTFQEFFGLSNCESTSQLDQATQVTETLG
MTHCCINPIIYAFVGEKFRSLFHIALGCRIAPLQKPVCGGPGVRPGKNV
KVTTQGLLDGRGKGKSIGRAPEASLQDKEGA

In some examples, the at least two proteins against which the two different inhibitors act are selected from the group consisting of Rac1, RAGE and CCR2.

In some examples, at least one of the at least two proteins is RAGE.

In some examples, at least one of the at least two protein is Rac1.

In some examples, at least one of the at least two proteins is CCR2.

In some examples, the combination comprises at least an inhibitor of the RAGE protein and an inhibitor of the Rac1 protein.

In some examples, the combination comprises at least an inhibitor of the RAGE protein and an inhibitor of the CCR2 protein.

In some examples, the combination comprises at least an inhibitor of the Rac1 protein and an inhibitor of the CCR2 protein.

In some examples, the combination comprises three inhibitors, each inhibitor being targeted to a protein of a different pathway associated with the retinal disease.

In some examples, the combination comprises at least one inhibitor of Rac1 protein, at least one inhibitor or RAGE protein and at least one inhibitor of CCR2 protein.

In some aspects, the present disclosure provides a combination comprising at least two inhibitors of respectively at least two proteins for use in one or more of inhibiting endothelial cells proliferation, inhibiting endothelial cells migration, inhibiting capillary sprouting, inhibiting retinal damage, treating retinal diseases or any combination thereof, wherein at least one of the at least two proteins is CCR2.

In some aspects, the present disclosure provides a combination comprising at least two inhibitors of respectively at least two proteins for use in one or more of inhibiting endothelial cells proliferation, inhibiting endothelial cells migration, inhibiting capillary sprouting, inhibiting retinal damage, treating retinal diseases or any combination thereof, wherein at least one of the at least two proteins is RAGE.

In some aspects, the present disclosure provides a combination comprising at least two inhibitors of respectively at least two proteins for use in one or more of inhibiting endothelial cells proliferation, inhibiting endothelial cells migration, inhibiting capillary sprouting, inhibiting retinal damage, treating retinal diseases or any combination thereof, wherein at least one of the at least two proteins is Rac1.

In some aspects, the present disclosure provides a combination comprising at least an inhibitor of the RAGE protein and an inhibitor of the Rac1 protein for use in one or more of inhibiting endothelial cells proliferation, inhibiting endothelial cells migration, inhibiting capillary sprouting inhibiting retinal damage, treating retinal diseases or any combination thereof.

In some aspects, the present disclosure provides a combination comprising at least an inhibitor of the RAGE protein and an inhibitor of the CCR2 protein for use in one or more of inhibiting endothelial cells proliferation, inhibiting endothelial cells migration, inhibiting capillary sprouting inhibiting retinal damage, treating retinal diseases or any combination thereof.

In some aspects, the present disclosure provides a combination comprising at least an inhibitor of the Rac1 protein and an inhibitor of the CCR2 protein for use in one or more of inhibiting endothelial cells proliferation, inhibiting endothelial cells migration, inhibiting capillary sprouting inhibiting retinal damage, treating retinal diseases or any combination thereof.

In some aspects, the present disclosure provides a combination comprising three inhibitors for use in one or more of inhibiting endothelial cells proliferation, inhibiting endothelial cells migration, inhibiting capillary sprouting inhibiting retinal damage, treating retinal diseases or any combination thereof, each inhibitor being targeted to a protein of a different pathway associated with the retinal disease.

In some aspects, the present disclosure provides a combination comprising at least one inhibitor of Rac1 protein, at least one inhibitor of RAGE protein and at least one inhibitor of CCR2 protein for use in one or more of inhibiting endothelial cells proliferation, inhibiting endothelial cells migration, inhibiting capillary sprouting inhibiting retinal damage, treating retinal diseases or any combination thereof.

In some examples, the CCR2 inhibitor affect binding of one or more CCR2 ligand to CCR2. In some examples, the CCR2 inhibitor inhibit interaction between CCR2 and one or more CCR2 ligands. In some examples, the CCR2 ligand is one or more of Monocyte chemoattractant protein-1 (MCP-1), Monocyte chemoattractant protein-2 (MCP-2), Monocyte chemoattractant protein-3 (MCP-3), Monocyte chemoattractant protein-4 (MCP-4), Monocyte chemoattractant protein-5 (MCP-5), or any combination thereof.

In some examples, the CCR2 inhibitor inhibit interaction between CCR2 and MCP-1.

In some examples, the inhibitor as described herein is an inhibitor of MCP1.

In some examples, the at least two proteins against which the two different inhibitors act are selected from the group consisting of Rac1, RAGE, CCR2 and MCP-1.

In some examples, the at least two proteins against which the two different inhibitors act are selected from the group consisting of Rac1, RAGE, CCR2/MCP-1.

In some examples, the at least two proteins against which the two different inhibitors act are selected from the group consisting of Rac1, RAGE and MCP-1.

MCP-1 protein also known as chemokine (CC-motif) ligand 2 (CCL2) is a member of chemokine subfamily and one of the key chemokines that regulate migration and infiltration of monocytes/macrophages.

In some examples, MCP-1 protein is human MCP-1.

In some examples, MCP-1 protein may comprise the amino acid sequence as denoted by NCBI Reference Sequence: NP_002973.1. In some examples,
the amino acid sequence of MCP-1 is as denoted by SEQ ID NO: 3.

SEQ ID NO:3 is provided by the following amino acid sequence (denoted by the single letter code)

MKVSAALLCLLLIAATFIPQGLAQPDAINAPVTCCYNFTNRKISVQRLA
SYRRITSSKCPKEAVIFKTIVAKEICADPKQKWVQDSMDHLDKQTQTPK
T

In some examples, at least one of the at least two proteins is MCP-1.

In some examples, at least one of the at least two proteins is RAGE.

In some examples, at least one of the at least two protein is Rac1.

In some examples, at least one of the at least two protein is CCR2.

In some examples, the combination comprises at least an inhibitor of the RAGE protein and an inhibitor of the MCP-1 protein.

In some examples, the combination comprises at least an inhibitor of the Rac1 protein and an inhibitor of the MCP-1 protein.

In some examples, the combination comprises three inhibitors, each inhibitor being targeted to a protein of a different pathway associated with the retinal disease.

In some examples, the combination comprises at least one inhibitor of Rac1 protein, at least one inhibitor or RAGE protein and at least one inhibitor of MCP-1 protein.

In some aspects, the present disclosure provides a combination comprising at least two inhibitors of respectively at least two proteins for use in one or more of inhibiting endothelial cells proliferation, inhibiting endothelial cells migration, inhibiting capillary sprouting, inhibiting retinal damage, treating retinal diseases or any combination thereof, wherein at least one of the at least two proteins is MCP-1.

In some aspects, the present disclosure provides a combination comprising at least two inhibitors of respectively at least two proteins for use in one or more of inhibiting endothelial cells proliferation, inhibiting endothelial cells migration, inhibiting capillary sprouting, inhibiting retinal damage, treating retinal diseases or any combination thereof, wherein at least one of the at least two proteins is RAGE.

In some aspects, the present disclosure provides a combination comprising at least two inhibitors of respectively at least two proteins for use in one or more of inhibiting endothelial cells proliferation, inhibiting endothelial cells migration, inhibiting capillary sprouting, inhibiting retinal damage, treating retinal diseases or any combination thereof, wherein at least one of the at least two proteins is Rac1.

In some aspects, the present disclosure provides a combination comprising at least an inhibitor of the RAGE protein and an inhibitor of the Rac1 protein for use in one or more of inhibiting endothelial cells proliferation, inhibiting endothelial cells migration, inhibiting capillary sprouting inhibiting retinal damage, treating retinal diseases or any combination thereof.

In some aspects, the present disclosure provides a combination comprising at least an inhibitor of the RAGE protein and an inhibitor of the MCP-1 protein for use in one or more of inhibiting endothelial cells proliferation, inhibiting endothelial cells migration, inhibiting capillary sprouting inhibiting retinal damage, treating retinal diseases or any combination thereof.

In some aspects, the present disclosure provides a combination comprising at least an inhibitor of the Rac1 protein and an inhibitor of the MCP-1 protein for use in one or more of inhibiting endothelial cells proliferation, inhibiting endothelial cells migration, inhibiting capillary sprouting inhibiting retinal damage, treating retinal diseases or any combination thereof.

In some aspects, the present disclosure provides a combination comprising three inhibitors for use in one or more of inhibiting endothelial cells proliferation, inhibiting endothelial cells migration, inhibiting capillary sprouting inhibiting retinal damage, treating retinal diseases or any combination thereof, each inhibitor being targeted to a protein of a different pathway associated with the retinal disease.

In some aspects, the present disclosure provides a combination comprising at least one inhibitor of Rac1 protein, at least one inhibitor or RAGE protein and at least one inhibitor of MCP-1 protein for use in one or more of inhibiting endothelial cells proliferation, inhibiting endothelial cells migration, inhibiting capillary sprouting inhibiting retinal damage, treating retinal diseases or any combination thereof.

The inhibitor also denoted at times antagonist, as used herein encompasses any molecule (compound, agent etc.) capable of binding to a protein or receptor and partially or completely blocking stimulation, decreasing or delaying activation or inactivating or downregulating the protein or receptor. In the context of the present disclosure, the antagonist or inhibitor may partially or completely block the activity of RAGE, Rac1, CCR2, MCP-1 or any combination thereof.

As appreciated, an inhibitor or an antagonist mediates the effect(s) by binding to the active site or to allosteric sites on any cognate protein (or receptor, in case applicable), or they may interact at unique binding sites not normally involved in the biological regulation of the cognate protein.

In some embodiments, the inhibitor may be a direct inhibitor. In some embodiments, the inhibitor may be a competitor inhibitor. A competitive antagonist directly and physically blocks access of the agonist to the protein.

In some other embodiments, the inhibitor is an allosteric inhibitor.

In some embodiments, the inhibitor is a negative allosteric modulator.

A negative allosteric modulator indirectly changes binding by interacting at a secondary site on the protein to diminish the ability of an agonist to bind to a primary site of a protein.

In some examples, the inhibitor may comprise a synthetic or natural inhibitor.

In some examples, the inhibitor may be one or more of a nucleic acid modulator, a protein modulator, a peptide modulator, an antibody or fragment thereof, peptidomimetic modulator, a small molecule modulator, or any combination thereof.

In some examples, the inhibitor is at least one of an aptamer, an antisense RNA, a single-stranded RNA (ssRNA), a double-stranded RNA (dsRNA) or any combination thereof.

In some examples, the inhibitor is a peptide.

In some examples, the inhibitor is a small molecule inhibitor (SMI).

Thus, in accordance with some aspects of present disclosure there is provided a combination for treating a retinal disease, the combination comprising at least two SMIs of respectively at least two proteins of different signaling pathways of the retinal disease.

A small molecule in the context of the present disclosure refers to a low molecular weight organic compound, having a molecular weight lower than 900 Daltons.

It should be noted that in accordance with the present disclosure, when referring to a small molecule it may encompasses one or more of the following as well as any combinations thereof: a solvate, a hydrate, a stereoisomer, a pharmaceutically acceptable prodrug, a pharmaceutically active metabolite, a pharmaceutically acceptable salt, a crystalline form, an amorphous form, a physiologically functional analogue, a physiologically functional derivative thereof or a combination thereof.

It should be noted that a solvate, a hydrate, a stereoisomer, a pharmaceutically acceptable prodrug, a pharmaceutically active metabolite, a pharmaceutically acceptable salt, a crystalline form, an amorphous form, a physiologically functional analogue, a physiologically functional derivative in the context of the present disclosure are considered to have similar biological or physiological activity as the small molecule to which they relate or any small molecule related thereof.

As noted herein, when the present disclosure relates to at least one inhibitor (antagonist) being a small molecule, it may encompass at least one of solvate, a hydrate, a stereoisomer, a pharmaceutically acceptable prodrug, a pharmaceutically active metabolite, a pharmaceutically acceptable salt, a crystalline form, an amorphous form, physiologically functional analogue, a physiologically functional derivative thereof or a combination thereof.

The term “solvate” refers to an aggregate of a molecule with one or more solvent molecules, such as hydrate, alcoholate (aggregate or adduct with alcohol), and the like.

The term “hydrate” refers to a compound formed by the addition of water. The hydrates may be obtained by any known method in the art by dissolving the compounds in water and recrystallizing them to incorporate water into the crystalline structure.

The term “stereoisomer” as used herein encompasses an “enantiomer”, the enantiomer refers to a compound that is superposable with respect to its counterpart only by a complete inversion/reflection (mirror image) of each other. The small molecule in accordance with the present disclosure encompass any enantiomers (i.e., R or S).

The term “prodrug” or “pharmaceutically acceptable prodrug” as used herein refers to a compound that may be converted under physiological conditions to the specified compound or to a pharmaceutically acceptable salt of such compound. Prodrugs may be useful for facilitating the administration of a parent drug.

The term “metabolite” or “pharmaceutically acceptable metabolite” as used herein refers to a compound that is formed under physiological conditions to of degrading and eliminating the compounds. Oxidative metabolite may an example.

The term “pharmaceutically acceptable salt” refers to salts derived from organic and inorganic acids of a compound described herein. Exemplary salts include, but are not limited to, sulfate, citrate, acetate, oxalate, chloride, hydrochloride, bromide, hydrobromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, camphorsulfonate, napthalenesulfonate, propionate, succinate, fumarate, maleate, malonate, mandelate, malate, phthalate, and pamoate. The term “pharmaceutically acceptable salt” as used herein also refers to a salt of a compound described herein having an acidic functional group, such as a carboxylic acid functional group, and a base. Exemplary bases include, but are not limited to, hydroxide of alkali metals including sodium, potassium, and lithium; hydroxides of alkaline earth metals such as calcium and magnesium; hydroxides of other metals, such as aluminum and zinc; ammonia, organic amines such as unsubstituted or hydroxyl-substituted mono-, di-, or tri-alkylamines, dicyclohexylamine; tributyl amine; pyridine; N-methyl, N-ethylamine; diethylamine; triethylamine; mono-, bis-, or tris-(2-OH—(C₁-C₆)-alkylamine), such as N, N-dimethyl-N-(2-hydroxyethyl)amine or tri-(2-hydroxyethyl)amine; N-methyl-D-glucamine; morpholine; thiomorpholine; piperidine; pyrrolidine; and amino acids such as arginine, lysine, and the like. The term “pharmaceutically acceptable salt” also includes hydrates of a salt of a compound described herein.

A crystalline and/or amorphous forms of the small compounds described herein include, for example, polymorphs, pseudopolymorphs, solvates, hydrates, unsolvated polymorphs (including anhydrates), conformational polymorphs, and amorphous forms of the compounds, as well as mixtures thereof.

The term “physiologically functional derivative” used herein relates to any physiologically acceptable derivative of a compound as described herein. The physiologically functional derivatives also include prodrugs of the compounds of the invention. As noted herein, such prodrugs may be metabolized in vivo to a compound of the invention. These pro-drugs may or may not be active themselves and are also an object of the present invention.

The term “derivative” in accordance with the small molecule of the present invention also encompasses chemically modified small molecule derived from a parent compound of the invention that differs from the parent compound by one or more elements, substituents and/or functional groups such that the derivative has the same or similar biological properties/activities as the parent compound.

The term “functional analog” used herein relates to any chemical entity that exhibits at least an inhibitory activity in at least one of the above screening assays, the activity of the analog can be the same or similar to that of the SMI to which it is analogous (herein the “parent SMI”). For the sake of clarity, when referring to a “parent SMI” it is to be understood to refer to an SMI that has been experimentally shown to be potentially effective in treating a retinal disease when tested in a combination as disclosed herein. The potential effect can be determined based on the above screening assays, and/or proven experimentally in in vivo models.

In some examples, the functional analog is a chemical derivative of the parent SMI, i.e., having at least one modification (e.g., deletion, substitution, insertion of a chemical group, isomerization etc.) with respect to the parent SMI and yet it has an inhibitory effect on the target protein being inhibited by the parent SMI. The inhibitory effect may not need to be to the same extent as that of the parent SMI and can be statistically greater, smaller, the same (as long as statistically, there is an inhibition).

In some examples, the functional analog may be structurally significantly different from the parent SMI (i.e., would not be considered a chemical derivative) and yet exhibit an inhibitory effect on the same protein inhibited by the parent SMI.

In some examples, the SMI is selected based on structural and/or physical criteria that allow their bioavailability.

In some examples, the SMI fulfill at least two, at least three, or even all four of the following criteria:

it has a molecular weight of less than 900 Da, at times, less than 600 Da; at times, of or below 500 Da; at times of or below 400 Da.

It has no more than 5 hydrogen bond donors;
it has no more than 10 hydrogen bond acceptors; and
it has a partition coefficient (referred to at times as Log P) not greater than 5; at times, not greater than 4.

In some examples, each of the at least two SMI fulfill at least two, at least three, or even all four criteria of Lipinski's Rule of Five.

In some examples, the combination comprises at least an SMI of the RAGE protein and at least an SMI of the CCR2 protein.

In some examples, the combination comprises at least an SMI of the Rac1 protein and at least an SMI of the CCR2 protein.

In some examples, the combination comprises at least an SMI of the RAGE protein and at least an SMI of the Rac1 protein.

In some examples, the combination comprises at least an SMI of the RAGE protein and at least an SMI of the MCP-1 protein.

In some examples, the combination comprises at least an SMI of the Rac1 protein and at least an SMI of the MCP-1 protein.

In some examples, the combination comprises three SMI, each SMI being targeted to a protein of a different pathway associated with the retinal disease.

In some examples, the combination comprises at least one SMI of Rac1 protein, at least one SMI or RAGE protein and at least one SMI of MCP-1 protein.

In some examples, the combination comprises at least one SMI of Rac1 protein, at least one SMI or RAGE protein and at least one SMI of CCR2 protein.

The selection of SMIs suitable for use in the combination disclosed here can be determined based on their activity in at least one of the following screening assays including proliferation assay, such as that using WST-1 reagent (testing cleavage of 4-[3-(4-Iodophenyl)-2-(4-nitro-phenyl)-2H-5-tetrazolio]-1,3-benzene sulfonate to formazan, hereinafter “WST-1 screening test”), endothelial cell migration assay (e.g. testing human umbilical vein endothelial cells (HUVECs) cell migration), and/or choroid sprouting assay.

In some examples, an SMI suitable for use in combination according to the present disclosure is one exhibiting activity in at least two of the above exemplary assays.

In some examples, an SMI suitable for use in the combination disclosed herein is one that is either absent of having a relatively low/minor in vitro apoptotic/necrotic effect on endothelial cells (e.g., HUVEC).

In some examples, an SMI suitable for use in the combination disclosed herein is one that inhibits HIF1α activity. HIF1α cavity can be determined by any method known in the art, for example, using the method described in the examples below.

The present disclosure is not limited to a specific Rac1 inhibitor.

According to some embodiments, the inhibitor may be directed to one or more binding sites of Rac1. The binding site may be determined by any known method in the field, for example by computational methods.

In some examples, the Rac1 inhibitor binds to the nucleotide-binding site of Rac1.

In some embodiments, the Rac1 inhibitor is at least one SMI.

In some embodiments, the Rac1 SMI is at least one of EHop-016, Z62954982, NSC23766 trihydrochloride, Rac1 Inhibitor V, EHT 1864, or a solvate, a hydrate, a stereoisomer, a pharmaceutically acceptable prodrug, a pharmaceutically active metabolite, a pharmaceutically acceptable salt, a crystalline form, an amorphous form, a physiologically functional analogue, a physiologically functional derivative thereof or a combination thereof.

Each of these Rac1 inhibitors is briefly discussed below.

EHop-016, is a small molecule having CAS #1380432-32-5 and chemical name N4-(9-Ethyl-9H-carbazol-3-yl)-N2-[3-(4-morpholinyl) propyl]-2,4-pyrimidinediamine. EHop-016 is represented by the following chemical structure denoted as Formula (I):

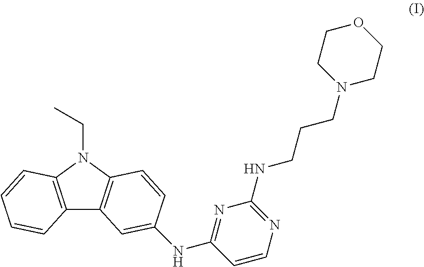 [SMILES: CCn1c2ccccc2c2cc(Nc3ccnc(NCCCN4CCOCC4)n3)ccc21]

Z62954982 (also denoted as Rac1 Inhibitor II), is a small molecule having CAS #109089312-1 and chemical name 3-((3,5-Dimethylisoxazol-4-yl) methoxy)-N-(4-methyl-3-sulfamoyl-phenyl)benzamide. Z62954982 is represented by the following chemical structure denoted as Formula (IV):

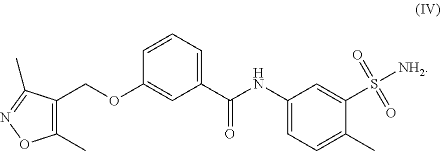 [SMILES: Cc1ccc(NC(=O)c2cccc(OCc3c(C)noc3C)c2)cc1S(N)(=O)=O]

NSC23766 trihydrochloride is a small molecule having CAS #1177865-17-6 and chemical name N6-[2-(4-Diethylamino-1-methyl-butylamino)-6-methyl-pyrimidin-4-yl]-2-methyl-quinoline-4,6-diamine trihydrochloride. NSC23766 trihydrochloride is represented by the following chemical structure denoted as Formula (V):

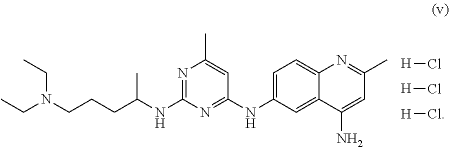

Rac1 Inhibitor V is a small molecule has chemical name 3-(2-Hydroxyphenyl)-N-(4-(1-piperidinylsulfonyl)phenyl)-1H-pyrazole-5-carboxamide. Rac1 Inhibitor V, is represented by the following chemical structure denoted as formula (VI)

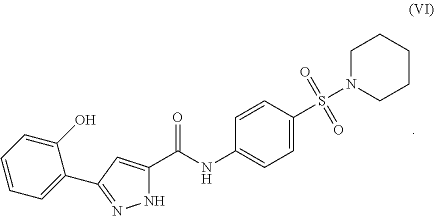 [SMILES: O=C(Nc1ccc(S(=O)(=O)N2CCCCC2)cc1)c1cc(-c2ccccc2O)n[nH]1]

EHT 1864, is a small molecule having CAS #754240-09-0 and chemical name 5-(5-(7-(Trifluoromethyl) quinolin-4-ylthio) pentyloxy)-2-(morpholinomethyl)-4H-pyran-4-one dihydrochlorideML141. EHT 1864 is represented by the following chemical structure denoted as formula (VII):

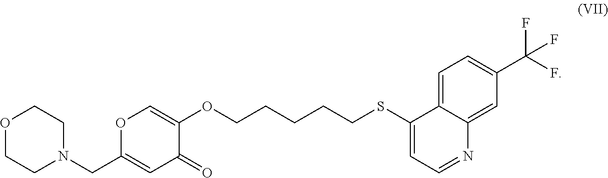 [SMILES: O=c1cc(CN2CCOCC2)occ1OCCCCCSc1ccnc2cc(C(F)(F)F)ccc12]

In some embodiments, the Rac1 inhibitor is at least one SMI having formula (I), (IV), (V) or a solvate, a hydrate, a stereoisomer, a pharmaceutically acceptable prodrug, a pharmaceutically active metabolite, a pharmaceutically acceptable salt, a crystalline form, an amorphous form, a physiologically functional analogue, a physiologically functional derivative thereof or a combination thereof.

In some embodiments, the Rac1 inhibitor is at least one SMI having formula (I), (IV), (V), (VI), (VII) or a solvate, a hydrate, a stereoisomer, a pharmaceutically acceptable prodrug, a pharmaceutically active metabolite, a pharmaceutically acceptable salt, a crystalline form, an amorphous form, a physiologically functional analogue, a physiologically functional derivative thereof or a combination thereof.

In some examples, the Rac1 inhibitor is a compound having Formula I.

In some examples, the combination comprises an SMI that is a functional analog of the inhibitor of Rac1 protein of Formula I.

The present disclosure is not limited to a specific RAGE inhibitor.

According to some embodiments, the inhibitor may be directed to one or more binding sites of RAGE. The binding site may be determined by any known method in the field, for example by computational methods.

In some examples, the RAGE inhibitor binds to the variable region of RAGE. In some examples, the RAGE inhibitor binds to the extracellular ligand-binding region of RAGE. In some examples, the RAGE inhibitor binds to the intracellular domain of RAGE.

In some embodiments, the RAGE inhibitor is at least one peptide.

In some embodiments, the RAGE inhibitor is at least one SMI.

In some examples, the RAGE SMI is at least one of Azeliragon, 4′-Methoxyresveratrol, FPS-ZM1, Tranilast or a solvate, a hydrate, a stereoisomer, a pharmaceutically acceptable prodrug, a pharmaceutically active metabolite, a pharmaceutically acceptable salt, a crystalline form, an amorphous form, a physiologically functional analogue, a physiologically functional derivative thereof or a combination thereof.

Each of these RAGE inhibitors is briefly discussed below.

Azeliragon (also known as TTP488; PF-04494700) having CAS #603148-36-3 and chemical name 3-[4-[2-butyl-1-[4-(4-chlorophenoxy)phenyl]-1H-imidazol-4-yl]phenoxy]-N,N-diethyl-1-propanamine. Azeliragon is represented by the following chemical structure denoted as Formula (II):

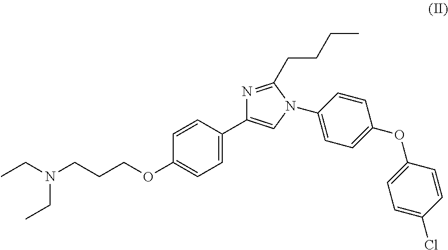 [SMILES: CCCCc1nc(-c2ccc(OCCCN(CC)CC)cc2)cn1-c1ccc(Oc2ccc(Cl)cc2)cc1]

4′-Methoxyresveratrol, having CAS #33626-08-3 and chemical name5-[(E)-2-(4-Methoxyphenyl) ethen-1-yl]benzene-1,3-diol. 4′-Methoxyresveratrol is represented by the following chemical structure denoted as Formula (VIII):

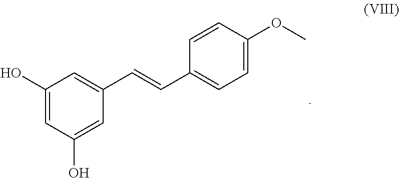 [SMILES: COc1ccc(/C=C/c2cc(O)cc(O)c2)cc1]

FPS-ZM1 having CAS #945714-67-0 and chemical name 4-Chloro-N-cyclohexyl-N-(phenylmethyl)benzamide. FPS-ZM1 is represented by the following chemical structure denoted as Formula (IX):

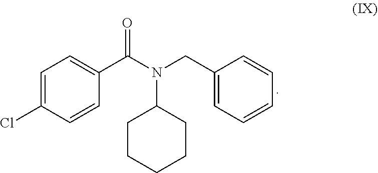 [SMILES: O=C(c1ccc(Cl)cc1)N(Cc1ccccc1)C1CCCCC1]

Tranilast has chemical name N-[3′,4′-dimethoxycinnamoyl]-anthranilic acid. Tranilast is represented by the following chemical structure denoted as formula (X)

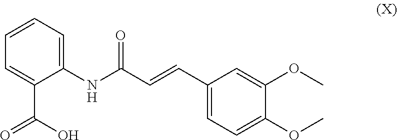 [SMILES: COc1ccc(/C=C/C(=O)Nc2ccccc2C(=O)O)cc1OC]

In some examples, the Rac1 inhibitor is at least one SMI having formula (II), (VIII), (IX) or a solvate, a hydrate, a stereoisomer, a pharmaceutically acceptable prodrug, a pharmaceutically active metabolite, a pharmaceutically acceptable salt, a crystalline form, an amorphous form, a physiologically functional analogue, a physiologically functional derivative thereof or a combination thereof.

In some examples, the RAGE inhibitor is a compound having the following Formula II.

In some examples, the combination comprises an SMI that is a functional analog of the inhibitor of RAGE protein of Formula II.

The present disclosure is not limited to a specific CCR2 inhibitor.

According to some embodiments, the inhibitor may be directed to one or more binding sites of CCR2 (including allosteric sites). In some examples, the CCR2 inhibitor is an allosteric antagonist of CCR2.

According to some embodiments, the inhibitor may be directed to one or more binding sites of MCP-1. The binding site may be determined by any known method in the field, for example by computational methods. In some examples, the CCR2 inhibitor is at least one of CCR2-RA-[R], PF-4136309, CCR2 antagonist 4 hydrochloride or a solvate, a hydrate, a stereoisomer, a pharmaceutically acceptable prodrug, a pharmaceutically active metabolite, a pharmaceutically acceptable salt, a crystalline form, an amorphous form, a physiologically functional analogue, a physiologically functional derivative thereof or a combination thereof.

Each of these CCR2 inhibitors is briefly discussed below.

CCR2-RA-[R] having CAS #512177-83-2 and a chemical name (R)-4-Acetyl-1-(4-chloro-2-fluorophenyl)-5-cyclohexyl-3-hydroxy-1,5-dihydro-2H-pyrrol-2-one. CCR2-RA-[R] is represented by the following chemical structure denoted as Formula (III)

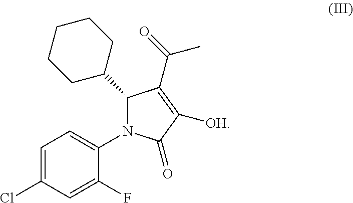 [SMILES: CC(=O)C1=C(O)C(=O)N(c2ccc(Cl)cc2F)[C@@H]1C1CCCCC1]

PF-4136309, having CAS #1341224-83-6 and a chemical name N-[2-[(3S)-3-[[4-hydroxy-4-(5-pyrimidin-2-ylpyridin-2-yl)cyclohexyl]amino]pyrrolidin-1-yl]-2-oxoethyl]-3-(trifluoromethyl)benzamide. PF-4136309 is represented by the following chemical structure denoted as Formula (XI)

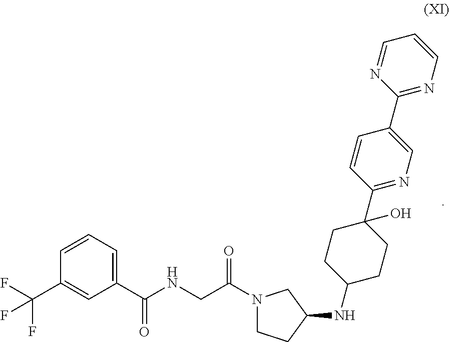 [SMILES: O=C(NCC(=O)N1CC[C@H](NC2CCC(O)(c3ccc(-c4ncccn4)cn3)CC2)C1)c1cccc(C(F)(F)F)c1]

CCR2 antagonist 4 hydrochloride (also known as Teijin compound 1 hydrochloride) having CAS #1313730-14-1 and a chemical name N-[2-[[(3R)-1-[(4-chlorophenyl)methyl]-3-pyrrolidinyl]amino]-2-oxoethyl]-3-(trifluoromethyl)benzamidehydrochloride. CCR2 antagonist 4 hydrochloride is represented by the following chemical structure denoted as Formula (XII)

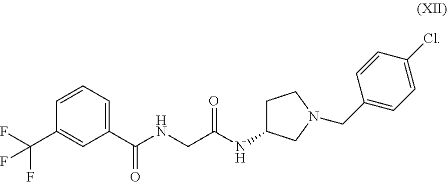 [SMILES: O=C(CNC(=O)c1cccc(C(F)(F)F)c1)N[C@@H]1CCN(Cc2ccc(Cl)cc2)C1]

In some examples, the CCR2 inhibitor is at least one SMI having formula (III), (XI), (XII) or a solvate, a hydrate, a stereoisomer, a pharmaceutically acceptable prodrug, a pharmaceutically active metabolite, a pharmaceutically acceptable salt, a crystalline form, an amorphous form, a physiologically functional analogue, a physiologically functional derivative thereof or a combination thereof.

In some examples, the CCR2 inhibitor is a compound having the following Formula III.

In some examples, the combination comprises an SMI that is a functional analog of the inhibitor of CCR2 protein of Formula III.

In some examples, the combination comprises at least two SMIs selected from (i) at least one SMI having Formula (I), (IV), (V), (VI) or (VII); (ii) at least one SMI having Formula (II), (VIII), (IX) or (X); (iii) at least one SMI having Formula (III), (XI) or (XII).

In some examples, the combination comprises at least two SMIs selected from (i) at least one SMI having Formula (I), (IV) or (V); (ii) at least one SMI having Formula (II), (VIII), (IX) or (X); (iii) at least one SMI having Formula (III), (XI) or (XII).

As shown in FIG. 5, combinations of SMIs showed an inhibitory effect with time.

In some examples, the combination comprises at least two SMIs having Formula (I) and Formula (II).

In some examples, the combination comprises at least two SMIs having Formula (I) and Formula (III).

In some examples, the combination comprises at least two SMIs having Formula (II) and Formula (II).

In some examples, the combination comprises at least three SMIs having Formula (I), Formula (II) and Formula (III).

In some examples, the combination of the present disclosure may be considered as a synergistic combination.

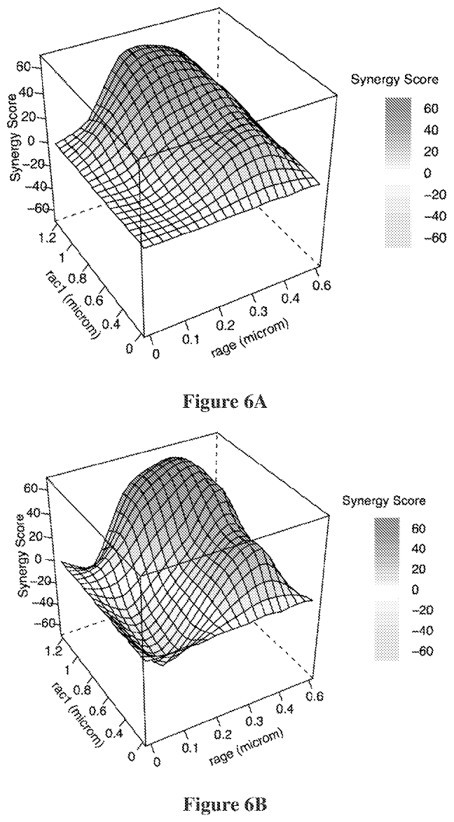

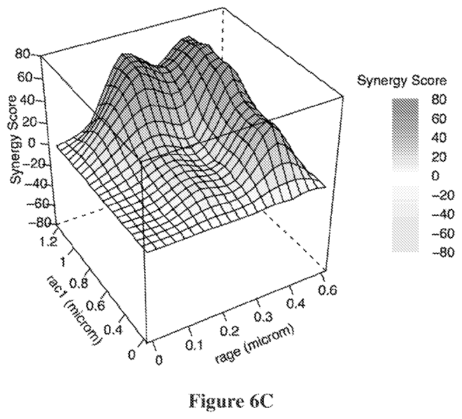

As shown in FIGS. 6A-6C, the combination of a Rac1 inhibitor and a RAGE inhibitor exhibited a synergistic effect in inhibiting endothelial cell proliferation over a broad concentration range.

The term synergistic effect or synergistic activity are interchangeably used herein.

A synergistic combination as used herein refers to a quantity of a combination comprising two or more SMIs that is statistically significantly more effective/active than the additive effects of the two or more SMIs when used individually (e.g., not in combination). A “synergistic amount” for a component in a combination is defined as an amount (e.g., concentration) providing a synergistic effect.

In some examples, the combination comprising a Rac1 inhibitor, and a RAGE inhibitor has a synergistic activity as compared to each of the Rac1 inhibitor and the RAGE inhibitor when used individually.

In some embodiments, each one of Rac1 inhibitor and the RAGE inhibitor is present in the combination in a concentration capable of providing a synergistic effect. In other words, and in accordance with some other embodiments, each one of the Rac1 inhibitor and the RAGE inhibitor is present in the combination in a synergistic amount.

In some examples, the combination comprising a Rac1 inhibitor and a CCR2 inhibitor has a synergistic activity as compared to each of the Rac1 inhibitor and the CCR2 inhibitor when used individually.

In some embodiments, each one of Rac1 inhibitor and CCR2 inhibitor is present in the combination in a concentration capable of providing a synergistic effect. In other words, and in accordance with some other embodiments, each one of the Rac1 inhibitor and the CCR2 inhibitor is present in the combination in a synergistic amount.

In some examples, the combination comprising a Rac1 inhibitor and a MCP-1 inhibitor has a synergistic activity as compared to each of the Rac1 inhibitor and the RAGE inhibitor when used individually. In some embodiments, each one of Rac1 inhibitor and MCP-1 inhibitor is present in the combination in a concentration capable of providing a synergistic effect. In other words, and in accordance with some other embodiments, each one of the Rac1 inhibitor and the MCP-1 inhibitor is present in the combination in a synergistic amount.

In some examples, the combination comprising a Rac1 inhibitor and a CCR2 inhibitor has a synergistic activity as compared to each of the Rac1 inhibitor and the CCR2 inhibitor when used individually. In some embodiments, each one of Rac1 inhibitor and CCR2 inhibitor is present in the combination in a concentration capable of providing a synergistic effect. In other words, and in accordance with some other embodiments, each one of the Rac1 inhibitor and the CCR2 inhibitor is present in the combination in a synergistic amount.

In some examples, the combination comprising a MCP-1 inhibitor, and a RAGE inhibitor has a synergistic activity as compared to each of the MCP-1 inhibitor and the RAGE inhibitor when used individually. In some embodiments, each one of RAGE inhibitor and MCP-1 inhibitor is present in the combination in a concentration capable of providing a synergistic effect. In other words, and in accordance with some other embodiments, each one of RAGE inhibitor and MCP-1 inhibitor is present in the combination in a synergistic amount.

In some examples, the combination comprising a CCR2 inhibitor, and a RAGE inhibitor has a synergistic activity as compared to each of the CCR2 inhibitor and the RAGE inhibitor when used individually. In some embodiments, each one of RAGE inhibitor and CCR2 inhibitor is present in the combination in a concentration capable of providing a synergistic effect. In other words, and in accordance with some other embodiments, each one of RAGE inhibitor and CCR2 inhibitor is present in the combination in a synergistic amount.

In some examples, the combination comprising a Rac1 inhibitor, a RAGE inhibitor and a MCP-1 inhibitor has a synergistic activity as compared to each of the Rac1 inhibitor, the RAGE inhibitor and the MCP-1 inhibitor when used individually. In some embodiments, each one of Rac1 inhibitor, the RAGE inhibitor and the MCP-1 inhibitor is present in the combination in a concentration capable of providing a synergistic effect. In other words, and in accordance with some other embodiments, each one of the Rac1 inhibitor, the RAGE inhibitor and the MCP-1 inhibitor is present in the combination in a synergistic amount.

In some examples, the combination comprising a Rac1 inhibitor, a RAGE inhibitor and a CCR2 inhibitor has a synergistic activity as compared to each of the Rac1 inhibitor, the RAGE inhibitor and the CCR2 inhibitor when used individually. In some embodiments, each one of Rac1 inhibitor, the RAGE inhibitor and the CCR2 inhibitor is present in the combination in a concentration capable of providing a synergistic effect. In other words, and in accordance with some other embodiments, each one of the Rac1 inhibitor, the RAGE inhibitor and the CCR2 inhibitor is present in the combination in a synergistic amount.

As detailed herein and shown in the Examples, the synergistic amount may be provided by a range of concentrations for each one of the inhibitors, such that various combinations of the inhibitors described herein provide a synergistic effect.

In accordance with some examples, the synergistic amount of a RAGE inhibitor may be a range of concentrations or may be a single concentration. In accordance with some examples, the synergistic amount of a Rac1 inhibitor may be a range of concentrations or may be a single concentration. In accordance with some examples, the synergistic amount of a MCP-1 inhibitor may be a range of concentrations or may be a single concentration.

In accordance with some examples, the synergistic amount of a RAGE inhibitor may be a range of concentrations or may be a single concentration. In accordance with some examples, the synergistic amount of a Rac1 inhibitor may be a range of concentrations or may be a single concentration. In accordance with some examples, the synergistic amount of a CCR2 inhibitor may be a range of concentrations or may be a single concentration.

In some examples, the synergistic combination comprises at least two SMIs selected from Formula (I), Formula (II) and Formula (III).

In some examples, the synergistic combination comprises at least SMIs represented by Formula (I) and Formula (II).

In some examples, the synergistic combination comprises at least SMIs represented by Formula (I) and Formula (III).

In some examples, the synergistic combination comprises at least SMIs represented by Formula (II) and Formula (III).

Synergism between at least two of the inhibitors may be based on the results obtained from the methods described herein, specifically methods described in the Examples below. For example, the synergistic effect of the inhibitors can be determined by the effect on endothelial cell proliferation as described in the Examples below. In some embodiments, the synergistic effect of the inhibitors on endothelial cell proliferation is determined as a synergistic combination using a ZIP model. In some embodiments, the synergistic effect of the inhibitors on endothelial cell proliferation is determined as a synergistic combination using a Loewe additivity model method. In some embodiments, the synergistic effect of the inhibitors on endothelial cell proliferation is determined as a synergistic combination using a HAS model.

The combination disclosed herein is effective against a variety of retinal diseases.

When referring to a retinal disease or an ocular disease it is to be understood to encompass any condition associated with the retina or choroid that can be caused by any one of angiogenesis, inflammation, trauma, oxidative stress, hypoxia, undesired immune response, diabetes, vascular occlusion, structural, atrophy, prematurity, toxicity, inherited degeneration, radiation, developmental retardation/defect, oncologic, hematologic, metabolic, vitamins deficiency, infectious, trauma, senile (aging) or different combination between them.

In some examples, the retinal disease comprises at least one condition selected from the group consisting of wet (neovascular) age-related macular degeneration (nAMD), diabetic retinopathy (DR, including proliferative diabetic retinopathy (PDR), and non-proliferative diabetic retinopathy (nPDR)), diabetic macular oedema (DMO), retinopathy of prematurity, radiation retinopathy, hypertensive retinopathy, myopic choroidal neovascularization (CNV), retinal vein occlusion (RVO), retinal artery occlusion (RAO), vasoproliferative tumor, coat's diseases, familial exudative vitreoretinopathy (FEVR), uveitis induced CNV, inherited retinal degeneration associated CNV, sickle cell retinopathy, idiopathic choroidal neovascularization, Irvine-Gass syndrome, choroidal hemangioma, dry age-related macular degeneration (AMD), retinal atrophy, carcinoma associated retinopathy (CAR), autoimmune induced retinopathy (AIR), central serous chorioretinopathy (CSCR), uveitis, inherited retinal degeneration (IRD), cystoid macular edema, proliferative vitreoretinopathy (PVR), Polypoidal choroidal vasculopathy (PCV).

In some examples, the retinal disease is a retinal vascular disease.

In some examples, the retinal vascular disease comprises at least wet (neovascular) age-related macular degeneration (nAMD).

In some examples, the retinal vascular disease comprises at least diabetic retinopathy (DR).

In some examples, the retinal vascular disease comprises at least proliferative diabetic retinopathy (PDR).

In some examples, the retinal vascular disease comprises at least non-proliferative diabetic retinopathy (NPDR).

In some examples, the retinal vascular disease comprises at least diabetic macular oedema (DMO).

In some examples, the retinal vascular disease comprises at least retinal vein occlusion (RVO).

In some examples, the retinal vascular disease comprises at least myopic choroidal neovascularization (CNV).

In some examples, the retinal vascular disease comprises at least retinopathy of prematurity (ROP).

In some examples, the retinal vascular disease comprises at least radiation retinopathy.

In some examples, the retinal vascular disease comprises at least vasoproliferative tumor.

In some examples, the retinal vascular disease comprises at least polypoidal choroidal vasculopathy (PCV)

In some examples, the retinal vascular disease comprises at least coat's disease.

In some examples, the retinal vascular disease comprises at least familial exudative vitreoretinopathy (FEVR).

In some examples, the retinal vascular disease comprises at least inherited retinal degeneration associated CNV.

In some examples, the retinal vascular disease comprises at least sickle cell retinopathy.

In some examples, the retinal vascular disease comprises at least choroidal hemangioma.

In some examples, the retinal vascular disease comprises at hypertensive retinopathy.

In some examples, the retinal vascular disease comprises at least idiopathic choroidal neovascularization (CNV).

In some examples, the retinal vascular disease comprises at least uveitis induced choroidal neovascularization (CNV).

In some examples, the retinal vascular disease comprises at least carcinoma associated retinopathy (CAR).

In some examples, the retinal vascular disease comprises at least autoimmune induced retinopathy (AIR).

In some examples, the retinal vascular disease comprises at least dry age-related macular degeneration.

In some examples, the retinal disease is a non-vascular retinal disease. Without being limited thereto, the non-vascular retinal disease one with inflammatory and/or oxidative involvement including, without being limited thereto, dry age-related macular degeneration (AMD), central serous chorioretinopathy (CSCR), uveitis (Idiopathic, immune, inflammatory and/or infectious), uveitis associated choroidal and/or retinal neovascularization, inherited retinal degeneration (IRD), toxic retinopathy, metabolic retinopathy, Irvine-Gass Syndrome, cystoid macular edema, and proliferative vitreoretinopathy (PVR).

The SMIs described herein can be formulated into suitable compositions for use. The at least one of the at least two SMIs of the combination disclosed herein can be formulated into a delivery system, preferably with a pharmaceutically acceptable carrier. Such delivery systems typically control the rate at which a drug is released and the location in the body where it is released. Some systems can control both.

In some embodiments, the delivery system is one or more of liposome, noisome, microsponge, microemulsion, microsphere, solid lipid nanoparticles (SLN), aerosol or combination thereof. In the context of the present disclosure, it is to be understood that a “pharmaceutically acceptable carrier” means a carrier that is useful in preparing a composition or formulation that is generally safe, non-toxic and neither biologically nor otherwise undesirable. The carrier is one that is acceptable for use on a living body, preferably mammals (humans and non-humans).

Some examples of suitable carriers or excipients for delivery of the combination disclosed herein include, without being limited thereto, polylactic acid (PLA), poly-lactic-co-glycolic acid (PLGA), polyvinyl alcohol (PVA), polyethyleneimine (PEI), lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, sterile water, syrup, and methyl cellulose.

The composition can additionally include lubricating agents such as talc, magnesium stearate, and mineral oil; wetting agents; emulsifying and suspending agents; preserving agents such as methyl- and propylhydroxy-benzoates; sweetening agents; and flavoring agents.

The composition disclosed herein can be formulated so as to provide quick, sustained or delayed release of the active ingredient after administration to the subject in need by employing procedures known in the art.

It is to be understood that the at least two SMIs are not necessarily formulated in a same delivery system, i.e., each can be delivered by a different type of delivery system or, if delivered by the same delivery principle (e.g., oral, topical, intravitreal, suprachoroidal), each can be formulated differently.

In some examples, at least one of the at least two SMI of the combination disclosed herein is formulated for oral delivery, e.g., for systemic delivery of the delivered SMI.

In some examples, at least one of the at least two SMI of the combination disclosed herein is formulated for intranasal delivery, e.g., as nasal spray, nasal drops, nasal ointment.

In some other examples, at least one of the at least two SMI of the combination disclosed herein is formulated for local administration, e.g., for topical delivery, e.g., as eye drops, eye ointment; and by local/intravitreal injection.

In yet some other examples, at least one of the at least two SMI of the combination disclosed herein is formulated for injection, e.g., intravenous (IV) injection, intramuscular (IM) injection.

In some examples, at least one of the at least two SMI is formulated for controlled delivery thereof. In some examples, the at least two SMI are each formulated for controlled delivery thereof.

In the context of the present disclosure, it is to be understood that “controlled delivery” denotes any one of slow release, delayed release, immediate/burst release, triggered release, and any other controlled delivery form as known to those versed in the pharmaceutical art.

In some examples, the at least two SMIs are formulated separately, i.e., in two different pharmaceutical compositions. In some examples, the two pharmaceutical compositions can be designed and/or dosed for sequential administration. In some other examples, the two pharmaceutical compositions can be designed and/or dosed for concomitant administration.

In some examples, the combination of the at least two SMIs are formulated in the same delivery system.

In some embodiments, the at least two inhibitors are administered (at times prior to administration) in a single unit dose; namely in a unit which is suitable for administration to the subject (human or non-human) as detailed herein below. The unit dose can contain an effective amount—a prescribed quantity of the at least two inhibitors sufficient to produce a therapeutic effect.

Thus, in accordance with some aspects, there is also disclosed a pharmaceutical composition comprising at least two inhibitors of respectively at least two proteins of different signaling pathways associated with at least a retinal disease and optionally at least one of pharmaceutically acceptable carrier/s, excipient/s, auxiliaries, and/or diluent/s. It should be noted that the pharmaceutical composition comprising the at least two inhibitors, e.g. SMIs as the active component, each one in an effective amount. In some examples, the pharmaceutical composition is formulated for topical application to the eye, e.g., as eye drops, or eye ointment. As used herein the term “topical administration”, or “topical application”, means directly laying on or spreading on an eye tissue, especially a cornea, or on tissues surrounding the eye. The topically administrable compositions may be formulated into a suitable formulation or composition with at least one carrier.

The at least one carrier may be selected from powders, oils, creams, foams, ointments, lotions, gels, pastes, mousiness, hydrogels or combination thereof. In some other embodiment, the composition is in the form of a solution, a suspension, a paste, a cream, a foam, gel or an ointment. In some embodiment, the composition is an ocular solution or an ocular suspension. In some embodiment, the composition is an aqueous solution or an aqueous suspension. In some embodiment, the composition is in the form of eye drops, eye spray or eye cream.

In some embodiments, the composition is in the form of eye drops of a suspension or solution. In some embodiments, the composition is applied to the eye in a form of topical drop. The eye drops may be in isotonic, pH-adjusted, sterile saline. Administration of the eye drops into the eye may be using a dropper, or a container with a dropper nozzle or a tube with a nozzle.

In some embodiments, the composition is an ocular solution. The term solution as used herein encompasses a range of viscosities, ranging from low viscosity solution to high viscosity solutions (forming a gel-like solution).

The pH of ocular composition is an important feature for controlling for example the ocular acceptability of the composition and the absorption of the compound across the cornea. Ideally the pH of the composition should be adjusted to maximize the chemical stability and/or absorption of the compounds (the first compound and the second compound). In some embodiments, the pH of the ocular composition is about 7.4 as this is the pH of tear fluid.

In some examples, the pharmaceutical composition is formulated for injection.

In some examples, the pharmaceutical composition is formulated using intravitreal biodegradable polymeric implant, or implanted device for sustained, slow release.

In some embodiments the pharmaceutical composition comprises the at least two inhibitors at a ratio selected from 50:1, 20:1, 10:1, 8:1, 7:1, 6:1, 5:1, 4:1, 3:1, 2:1, 1:1, 1:1.5, 1:2, 1:2.5, 1:3, 1:4, 1:5. 1:6, 1:7, 1:8, 1:10, 1:20 or 1:50. The disclosed combination is suitable for use in a method of treating a subject having, being susceptible of having or predisposed to have a retinal disease.

Hence, in accordance with some accepts, a method for one or more of inhibiting endothelial cells proliferation, inhibiting endothelial cells migration, inhibiting capillary sprouting, inhibiting retinal damage, the method comprising contacting retina cells with at least SMIs of respectively at least two proteins of different signaling pathways of retinal disease.

The term “contacting” as used herein, means to bring, put, incubate or mix together. As such, a first item is contacted with a second item when the two items are brought or put together, e.g., by touching them to each other or combining them. In the context of the present invention, the term “contacting” includes all measures or steps, which allow interaction between the compounds of the invention and the cells or subjects to be modulated, as specified herein after.

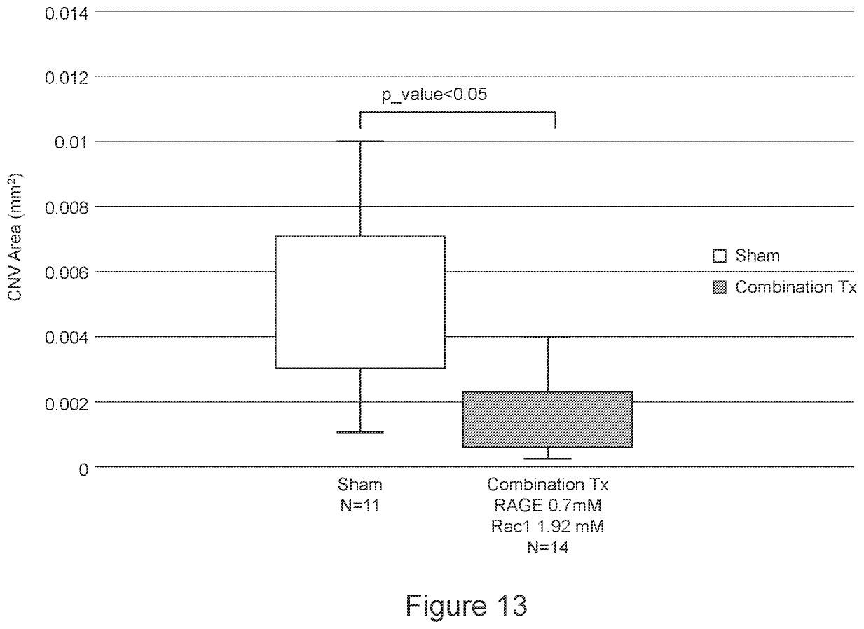

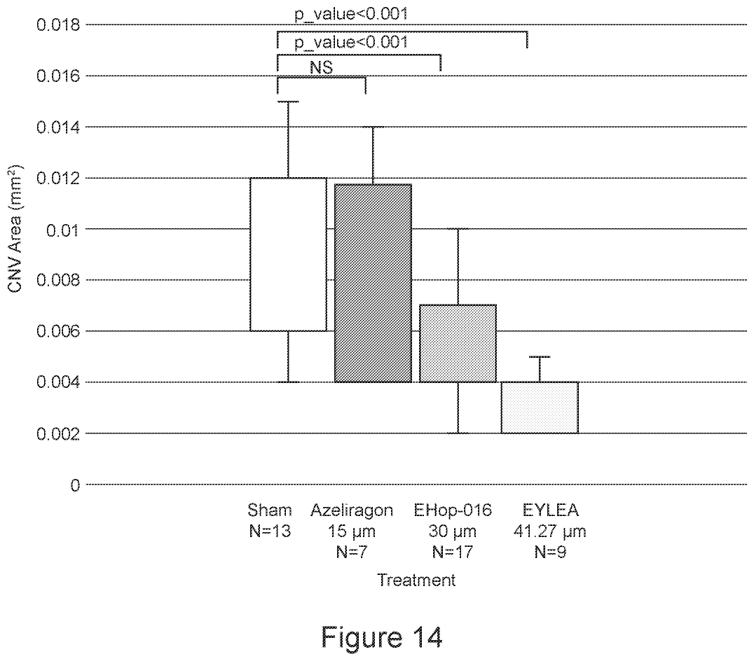

As shown in the Examples below, each one of the SMIs described herein as well as combinations were highly effective in in vivo studies. Specifically, as shown in FIGS. 12A and 12B, the combination of Azeliragon and EHop-016 inhibited formation of choroidal neovascularization. In addition, as shown in FIG. 13, CNV area, was significant lower in the mice treated with intravitreal injections of combinations of Azeliragon and EHop-016. Interestingly, as shown in FIG. 14, CNV area measured for the mice treated with Ehop-016, was comparable to the dramatic effect received from Aflibercept injection.

In some other aspects, it is provided a method of treating, inhibiting, arresting or delaying a retinal disease, the method comprises administering to a subject a therapeutically effective amount of at least two SMIs of respectively at least two proteins of different signaling pathways of retinal disease.

In some examples, the method disclosed herein comprises administering to a subject in need of treatment an amount of at least two SMIs of respectively at least two proteins of different signaling pathways of retinal disease, the amount being effective to provide said treatment.

In some examples, the method comprising administering to the subject two inhibitors of two or more of Rac1 protein, RAGE protein, CCR2 protein.

In some examples, the method comprising administering to the subject two inhibitors of two or more of Rac1 protein, RAGE protein, MCP-1 protein.

In some examples, the method comprising administering to the subject at least two SMIs selected one or more of: (i) at least one SMI having Formula (I), (IV), (V), (VI) or (VII); (ii) at least one SMI having Formula (II), (VIII), (IX) or (X); (iii) at least one SMI having Formula (III), (XI) or (XII).

In some examples, the method comprising administering to the subject at least two SMIs having Formula (I) and Formula (II).

In some examples, the method comprising administering to the subject at least two SMIs having Formula (I) and Formula (III).

In some examples, the method comprising administering to the subject at least two SMIs having Formula (II) and Formula (II).

In some examples, the method comprising administering to the subject at least three SMIs having Formula (I), formula (II) and Formula (III).

In some examples, the method may be applicable for treating at least one condition selected from the group consisting of wet (neovascular) age-related macular degeneration (nAMD), diabetic retinopathy (DR, including proliferative diabetic retinopathy (PDR), and non-proliferative diabetic retinopathy (nPDR)), diabetic macular oedema (DMO), retinopathy of prematurity, radiation retinopathy, hypertensive retinopathy, myopic choroidal neovascularization (CNV), retinal vein occlusion (RVO), retinal artery occlusion (RAO), vasoproliferative tumor, coat's diseases, familial exudative vitreoretinopathy (FEVR), uveitis induced CNV, inherited retinal degeneration associated CNV, sickle cell retinopathy, idiopathic choroidal neovascularization, Irvine-Gass syndrome, choroidal hemangioma, dry age-related macular degeneration (AMD), retinal atrophy, carcinoma associated retinopathy (CAR), autoimmune induced retinopathy (AIR), central serous chorioretinopathy (CSCR), uveitis, inherited retinal degeneration (IRD), cystoid macular edema, proliferative vitreoretinopathy (PVR), Polypoidal choroidal vasculopathy (PCV).

In some examples, the method of the disclosure may be applicable for treating AMD.

In some examples, the method of the disclosure may be applicable for treating wet-AMD.

In some examples, the method of the disclosure may be applicable for treating DR.

In some examples, the method of the disclosure may be applicable for treating PDR.

In some examples, the method of the disclosure may be applicable for treating NPDR.

In some examples, the method of the disclosure may be applicable for treating DMO.

In some examples, the method of the disclosure may be applicable for treating ROV.

In some examples, the method of the disclosure may be applicable for treating RAO.

In some examples, the method of the disclosure may be applicable for treating CNV.

In some examples, the method of the disclosure may be applicable for treating ROP.

In some examples, the method of the disclosure may be applicable for treating radiation retinopathy.

In some examples, the method of the disclosure may be applicable for treating vasoproliferative tumor.

In some examples, the method of the disclosure may be applicable for treating PCV).

In some examples, the method of the disclosure may be applicable for treating coat's disease.

In some examples, the method of the disclosure may be applicable for treating FEVR.

In some examples, the method of the disclosure may be applicable for treating inherited retinal degeneration associated CNV.

In some examples, the method of the disclosure may be applicable for treating sickle cell retinopathy.

In some examples, the method of the disclosure may be applicable for treating choroidal hemangioma.

In some examples, the method of the disclosure may be applicable for treating hypertensive retinopathy.

In some examples, the method of the disclosure may be applicable for treating idiopathic choroidal neovascularization (CNV).

In some examples, the method of the disclosure may be applicable for treating uveitis induced choroidal neovascularization (CNV).

In some examples, the method of the disclosure may be applicable for treating CAR.

In some examples, the method of the disclosure may be applicable for treating AIR.

In some examples, the method of the disclosure may be applicable for treating dry age-related macular degeneration.

In some examples, the method of the disclosure may be applicable for treating a non-vascular retinal disease.

In some examples, for the purpose of treating a retinal disease in a subject in need of the treatment, the at least two inhibitors, e.g. SMIs, as defined herein, are administered concomitantly. Thus, the method disclosed herein comprises concomitant administration of the at least two inhibitors, e.g. SMIs.

In some examples, the at least two inhibitors, e.g. SMIs are administered sequentially. Thus, the method disclosed herein comprises sequential administration of the at least two inhibitors, e.g. SMIs.

It should be noted that when the at least two inhibitors, e.g. SMIs are administered separately, the time between administration of one SMI to at least one other SMI can be minutes, hours, days, weeks.

Finally, disclosed herein is a package or kit comprising a first inhibitor, e.g. a SMI targeted against a protein of a signaling pathway of a retinal disease; at least one additional inhibitor, e.g. SMI being different from said first inhibitor and targeted at a different protein of a signaling pathway of said retinal disease; and instructions for use of a combination of said first inhibitor and said at least one additional inhibitor for treating said retinal disease.

The first SMI in the kit is preferably contained separately from the at least one additional SMI. The first and the additional SMIs are as defined hereinabove with respect to the at least two SMIs for use in the disclosed combination.

In some embodiments, the different reservoirs are different syringes or different formulation containers comprising the actives in solid or liquid or solution forms.

In some embodiments, the kit comprise a container means for containing separate compositions; such as a divided bottle or a divided foil packet.

In some embodiments, the kit includes directions for the administration of the separate components. It should be understood that such a kit is advantageous when the at least two SMIs are preferably administered in different dosage forms, are administered at different dosage intervals, or when titration of the individual components of the combination is desired by the prescribing physician.

It should be appreciated that each of the multiple components of the kit may be administered simultaneously.

Alternatively, each of said multiple dosage forms may be administered sequentially in either order.

Further, the kits described herein can include a composition as described, or in separate multiple dosage unit forms, as an already prepared dosage form ready for administration or, alternatively, can include the composition as described as a solid pharmaceutical composition that can be reconstituted with a solvent to provide a liquid oral dosage form.

When the kit includes a solid pharmaceutical composition that can be reconstituted with a solvent to provide a liquid dosage form (e.g., for oral administration), the kit may optionally include a reconstituting solvent.

In some embodiments, the kit comprises the at least two SMIs in the same composition, e.g., the pharmaceutical composition of the present disclosure.

In some embodiments, the kit comprising instructions for the at least two SMIs in a combination for use in one or more of inhibiting endothelial cells proliferation, inhibiting endothelial cells migration, inhibiting tube formation, inhibiting capillary sprouting inhibiting retinal damage, treating retinal diseases or any combination thereof, in a subject in need thereof.

In accordance with some further aspects, the present disclosure provides use of at least two SMIs in the preparation of a pharmaceutical composition or a combination as described herein.

In the context of the present disclosure, the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As noted above, the invention provides methods for treating disorders specified above. In the context of the present disclosure, the terms “treating”, “treatment”, and the like are used herein to refer to refers to the administering of a therapeutic amount of the combination, composition or kits of the present invention which is effective to improve one or more undesired symptoms associated with a disease or condition as described herein, i.e. obtaining a desired pharmacological and physiological effect. The effect may be prophylactic in terms of preventing or partially preventing a disease, symptom or condition thereof and/or may be therapeutic in terms of a partial or complete cure of a disease, condition, symptom or adverse effect attributed to the disease. The term “treatment”, as used herein, covers any treatment of a retinal disease, preferably in a mammal, further particularly a human, and includes: (a) preventing the disease from occurring in a subject which may be predisposed to the disease but has not yet been diagnosed as having it, i.e., causing the clinical symptoms of the disease not to develop in a subject that may be predisposed to the disease but does not yet experience or display symptoms of the disease; (b) inhibiting the disease, i.e., arresting or reducing the development of the disease or its clinical symptoms; or (c) relieving the disease, i.e., causing regression of the disease and/or its symptoms or conditions. The present disclosure is directed towards treating a patient's suffering from a retinal disease. The present disclosure is involved in preventing, inhibiting, or relieving adverse effects attributed to retinal disease.

As indicated above, the method of the invention involves the administration of a therapeutically effective amount of each one of the inhibitors described herein. The “effective amount” or “therapeutically effective” for purposes disclosed herein indicates that the amount of formulation is effective to treat, inhibit or delay one or more symptoms of a disease as described herein. The “amount effective” or “therapeutically effective amount” for purposes herein is determined by such considerations as may be known in the art. The amount must be effective to achieve the desired therapeutic effect as described above, i.e., treating retinal disease, depending, inter alia, on the type and severity of the disease to be treated and the treatment regime. The amount is typically determined in appropriately designed clinical trials (dose range studies) and the person versed in the art will know how to properly conduct such trials in order to determine the effective amount. As generally known, an amount effective for the desired treatment depends on a variety of factors including the affinity of the SMI to the protein, its distribution profile within the body, a variety of pharmacological parameters such as half-life in the body, on undesired side effects, if any, on factors such as age and gender, etc.

As used herein, “disease”, “disorder”, “condition” and the like, as they relate to a subject's health, are used interchangeably and have meanings ascribed to each and all of such terms. It is understood that the interchangeably used terms “associated” and “related”, when referring to pathologies herein, mean diseases, disorders, conditions, or any pathologies which at least one of: share causalities, co-exist at a higher than coincidental frequency, or where at least one disease, disorder, condition or pathology causes a second disease, disorder, condition or pathology.

As used herein, the term “subject” refers to a living organism that is treated with the formulation as described herein, including, but not limited to, any mammal, such as a human.

The terms “inhibition”, “moderation”, “reduction” or “attenuation” as referred to herein, relate to the reduction for example in expression/level of at least one CC chemokine by any one of about 1% to 99.9%, specifically, about 1% to about 5%, about 5% to 10%, about 10% to 15%, about 15% to 20%, about 20% to 25%, about 25% to 30%, about 30% to 35%, about 35% to 40%, about 40% to 45%, about 45% to 50%, about 50% to 55%, about 55% to 60%, about 60% to 65%, about 65% to 70%, about 75% to 80%, about 80% to 85% about 85% to 90%, about 90% to 95%, about 95% to 99%, or about 99% to 99.9%.

As used herein, the forms “a”, “an” and “the” include singular as well as plural references unless the context clearly dictates otherwise. For example, the term “SMI” includes one or more small molecule inhibitors which are capable of specifically inhibiting a protein that is involved in a signaling pathway related to/associated with a retinal disease.

Further, as used herein, the term “comprising” is intended to mean that the combination, composition, method and/or kit include the recited SMIs, but not excluding other elements, such as physiologically acceptable carriers and excipients as well as other active agents. The term “consisting essentially of” is used to defines that the combination, composition, method and/or kit disclosed herein include the recited elements but exclude other elements that may have an essential significance on treatment of the retinal disease. “Consisting of” shall thus mean excluding more than trace elements of other elements. Embodiments defined by each of these transition terms are within the scope of this disclosure.

Further, all numerical values, e.g. when referring the amounts or ranges of the elements constituting the combination, composition, method and/or kit comprising defined SMIs are approximations which are varied (+) or (−) by up to 20%, at times by up to 10% of from the stated values. It is to be understood, even if not always explicitly stated that all numerical designations are preceded by the term “about”.

It should be noted that various embodiments of this invention may be presented in a range format. The description of a range should be considered to have specifically disclosed all the possible sub ranges as well as individual numerical values within that range. For example, description of a range such as from 1 to 6 or between 1 and 6 should be considered to have specifically disclosed sub ranges such as from 1 to 3, from 1 to 4, from 1 to 5, from 2 to 4, from 2 to 6, from 3 to 6 etc., as well as individual numbers within that range, for example, 1, 2, 3, 4, 5, and 6.

It should be further noted that the various embodiments and examples detailed herein in connection with various aspects of the invention may be applicable to one or more aspects disclosed herein. It should be further noted that any embodiment described herein, for example, related to combinations, may be applied separately or in various combinations. Various embodiments and aspects of the present invention as delineated hereinabove and as claimed in the claims section below find experimental support in the following examples. The phrases “in another embodiment” or any reference made to embodiment as used herein do not necessarily refer to different embodiment, although it may. Thus, various embodiments of the invention can be combined (from the same or from different aspects) without departing from the scope of the invention.

The invention will now be exemplified in the following description of experiments that were carried out in accordance with the invention. It is to be understood that these examples are intended to be in the nature of illustration rather than of limitation. Obviously, many modifications and variations of these examples are possible in light of the above teaching. It is therefore, to be understood that within the scope of the appended claims, the invention may be practiced otherwise, in a myriad of possible ways, than as specifically described hereinbelow.

### DESCRIPTION OF SOME NON-LIMITING EXAMPLES

### Materials and Methods

### Materials

Human Umbilical Vein Endothelial Cell line (HUVECs; Lonza, Cat #C2519A), Human fibroblast cell line and Human embryonic kidney 293 (HEK-293) cell line were both given by Prof. Dror Sharon Laboratory, Hadassah Medical Center, Jerusalem, Israel.
Cell proliferation reagent WST-1 was purchased from Sigma-Aldrich.
Matrigel™ matrix was purchased from Getter-Biomed.
EGM™-2 was purchased from Lonza Bioscience Solutions.
Annexin V-FITC Kit Cat. #130-092-052, Miltenyi Biotec.

### Methods

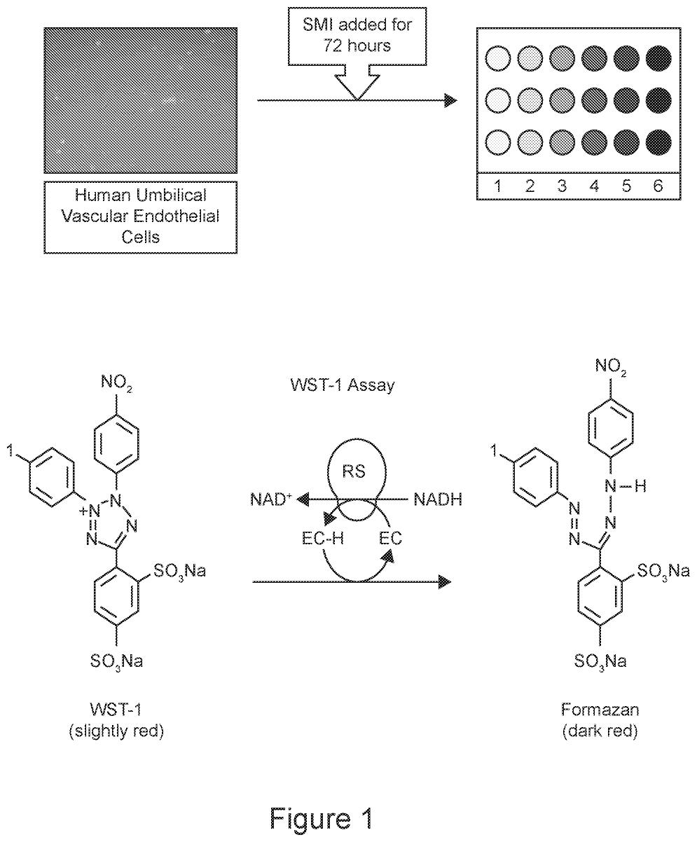

Cell proliferation assay—The principle of cell proliferation assay is illustrated in FIG. 1. Briefly, HUVECS were seeded and maintained in EGM™-2 (Lonza Bioscience Solutions) medium for 24 hours. The cells were then treated with various dosages of SMI for designated time periods. Each experiment included a DMSO-treated control group. Tetrazolium salt was added according to the manufacturer's instructions and formation of formazan dye was quantified by measuring the absorbance at 440 nm using spectrophotometer. The formation of formazan dye is directly proportional to number of living cells.

Analysis of combined synergistic effect-Analysis of combined SMIs effect was determined from the results of the cell proliferation assay after 72 hours. SMIs synergy was determined by three synergy models.
1. The Zero Interaction Potency (ZIP) model is a reference model for evaluating the effect of combination of two drugs by comparing the change in the potency of the dose-response curves between individual drugs and their combinations. A zip score higher than 10 is indicative of a synergistic combination, a zip score of between −10 and 10 is indicative of an additive combination and a zip score lower than −10 is indicative of an antagonistic combination.
2. The Loewe additivity model: is based on the assumption that two compounds have the same mechanism of action and therefore one compound can be a substitute for another. In case of no interaction between the compounds, the dose combinations will have the same response and will follow the equation: x1/f1+x2/f2=1. Values below 1 will represent synergism and above 1 antagonism. (Lederer, S., Dijkstra, T. M. and Heskes, T., 2018. Additive dose response models: explicit formulation and the Loewe additivity consistency condition. Frontiers in pharmacology, 9, p. 31.)
3. The HAS model states that the expected combination effect equals to the higher effect of individual drugs, i.e. yHSE=max (y1, y2). Therefore, any additional effect over the higher single drug will be considered as a HSA synergy.

Annexin V/propidium iodide (PI) assay—3×10³ cells/well of HUVECs were seeded in EGM™-2 medium for 24 hours. The cells were then treated with various dosages and combinations of SMI for 72 hours in addition to a control group treated with DMSO. Then, cells viability was measured using Annexin V-FITC Kit according to the manufacturer's instructions. Cells were stained with Annexin V-FITC and PI and analyzed by flow cytometry.

Endothelial cell migration assay (scratch test)—5×10⁴ cells/200 μl of HUVECs were cultured in 96 wells Incucyte® ImageLock 96-well microplates plates to near confluence in EGM™-2 medium for 24 hours. Afterwards, a homogeneous, 700-800-micron wide scratch wounds were created using Incucyte® WoundMaker. Different SMIs concentrations vs. DMSO (solvent) were added. Serial images for 26 hours of the migrating cells were obtained hourly using a live cell imaging system (Incucyte®).

Western blot—5×10⁴ HUVEC cells were seeded in 10 cm dish in EGM™-2 medium for 24 hours. The cells were then treated with various dosages and combinations of SMI for 72 hours in addition to a control group treated with DMSO. Cells were lysed in WCE (whole-cell extraction) buffer [25 mM Hepes, pH 7.7, 0.3 M NaCl, 1.5 mM MgCl2, 0.2 mM EDTA, 0.1% Triton X-100, 0.5 mM DTT (dithiothreitol), 20 mM 2-glycerophosphate, 0.1 mM Na3VO4, 100 μg/ml PMSF and protease inhibitor cocktail (Sigma-Aldrich, 1:100 dilution)]. The proteins were then separated by SDS/PAGE (10% gel), followed by transfer on to a nitrocellulose membrane. Blots were blocked in 5% (w/v) non-fat dried skimmed milk powder in PBS and washed in PBS. Proteins were detected using primary antibodies (Cell Signal, Anti-beta actin (CST-4970S) and anti-Hif1α (CST-14179S)) and corresponding HRP (horseradish peroxidase)-conjugated secondary antibodies obtained from Cell Signal (anti-rabbit CST-7074S).

Choroid sprouting assay—Choroid-sclera complex from C57BL/6J mice was gently dissected along with retinal pigment epithelium (RPE) and were embedded in 30 μl of growth factor-reduced Matrigel™ (BD Biosciences, Cat. 354230) in 24-well plates. Plates were then incubated for 10 minutes in 37° C., in a 5% CO₂ cell culture incubator without medium to solidify the Matrigel™. Medium (250 μl) containing ECGM (C-22010, PromoCell, Germany), 2.5% supplement mix (C-9215, PromoCell, Germany), 5% FCS, 1/100 penicillin-streptomycin, and 1/100 glutamine was added to each well. Fresh medium and SMIs were replaced every two days and grown for 7 days (x3 time medium replaced). Five different treatments were included: control without DMSO solvent, control with DMSO solvent, 0.55 μM of RAGE inhibitor Azeliragon, 0.70 M of Rac1 inhibitor EHop-016 and a combination of 0.55 μM of Azeliragon (RAGE inhibitor) combined with 0.70 μM of EHop-016 (Rac1 inhibitor); Images were obtained and quantified using ImageJ software.

Laser-Induced CNV Model—CNV was induced in C57BL/6J mice using argon green ophthalmic laser (120 MW, 50 μm, 0.1 sec) applied under fundoscopy to rupture Bruch's membrane. The mice treated at day 0 and 3 with intravitreal injections of Azeliragon (0.7 mM) and Ehop-016 (1.92 mM) combination vs. Sham. The injections were performed at the superotemporal sclera, where a perforating sclerotomy was made with 27-gauge (G) needle at approximately 1-2 mm from the limbus. Then, 1-2 μl of SMIs (solved in 5% Cremophor: 5% ethanol: 90% saline) vs. Sham were injected through the sclerotomy, under fundoscopy using 34 g microsyringe (Hamilton, Benfleet, UK).

Quantification of CNV—Eyes were enucleated, treated according to standard protocols. Choroid-RPE flat mounts were fixed for 1 h in 4% PFA and suspended overnight in Isolectin GS-IB4 Alexa Fluor 594 staining solution (Molecular Probes, Eugene, OR) containing 200 mM NaN₃ and 1 mM CaCl₂). The flat mounts were then washed six times for 20 min each in PBS and mounted on a slide using mounting medium. The area of CNV surrounding each laser injury was measured using ImageJ software.

### Example 1: Identification of Small Molecule Inhibitors Aimed Against Key Target Proteins Involved in Multiple Etiologies of Retinal Angiogenesis

The following compounds were tested for their inhibitory activity. The compounds were selected following Lipinski's rule of five to choose compounds with drug-like properties. In addition, compounds with relatively low inhibitory concentration (IC₅₀) which were used in other different in vitro and in vivo systems in order to increase the chances for activity.

TABLE 1
Selected SMIs aimed against three key target proteins

| | MW | | |
|---|---|---|---|
| Inhibitor | (Da) | Formula | Structure |
| CCR2 antagonist 4 hydrochloride | 476.32 | C₂₁H₂₂Cl₂F₃N₃O₂ Formula (XII) | 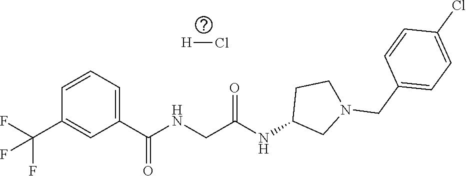 [SMILES: O=C(CNC(=O)c1cccc(C(F)(F)F)c1)N[C@@H]1CCN(Cc2ccc(Cl)cc2)C1 ; Cl] |
| PF-4136309 | 568.59 | C₂₉H₃₁F₃N₆O₃ Formula (XI) | 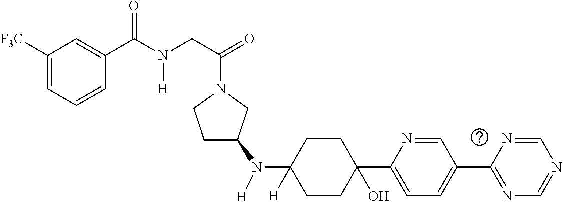 [SMILES: O=C(NCC(=O)N1CC[C@H](NC2CCC(O)(c3ccc(-c4ncncn4)cn3)CC2)C1)c1cccc(C(F)(F)F)c1] |
| CCR2-RA-[R] | 351.80 | C₁₈H₁₉ClFNO₃ Formula (III) | 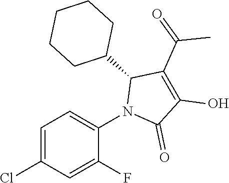 [SMILES: CC(=O)C1=C(O)C(=O)N(c2ccc(Cl)cc2F)[C@@H]1C1CCCCC1] |
| FPS-ZM1 | 327.85 | C₂₀H₂₂ClNO Formula (IX) | 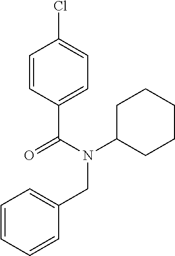 [SMILES: O=C(c1ccc(Cl)cc1)N(Cc1ccccc1)C1CCCCC1] |
| Azeliragon | 532.12 | C₃₂H₃₈ClN₃O₂ Formula (II) | 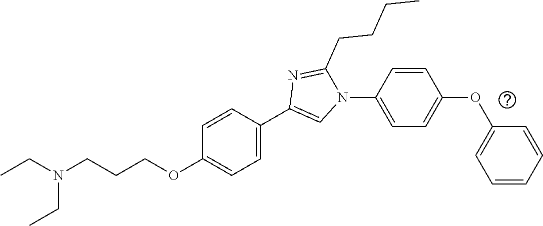 [SMILES: CCCCc1nc(-c2ccc(OCCCN(CC)CC)cc2)cn1-c1ccc(Oc2ccccc2)cc1] |
| 4′- Methoxy- resveratrol | 242.27 | C₁₅H₁₄O₃ Formula (VIII) | 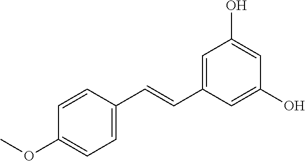 [SMILES: COc1ccc(/C=C/c2cc(O)cc(O)c2)cc1] |
| NSC 23766 trihydrochloride | 530.96 | C₂₄H₃₈Cl₃N₇ Formula (V) | 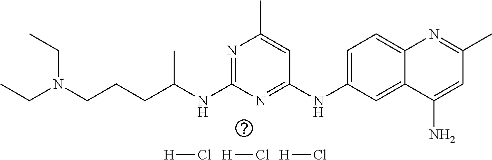 |
| Z62954982 | 415.46 | C₂₀H₂₁N₃O₅S Formula (IV) | 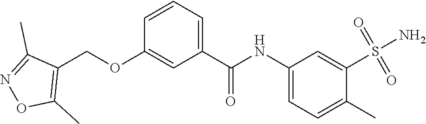 [SMILES: Cc1ccc(NC(=O)c2cccc(OCc3c(C)noc3C)c2)cc1S(N)(=O)=O] |
| EHop-016 | 430.55 | C₂₅H₃₀N₆O Formula (I) | 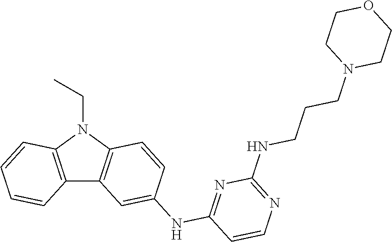 [SMILES: CCn1c2ccccc2c2cc(Nc3ccnc(NCCCN4CCOCC4)n3)ccc21] |

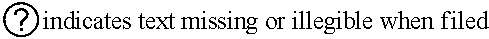

### Example 2: Proliferation Inhibition Assay

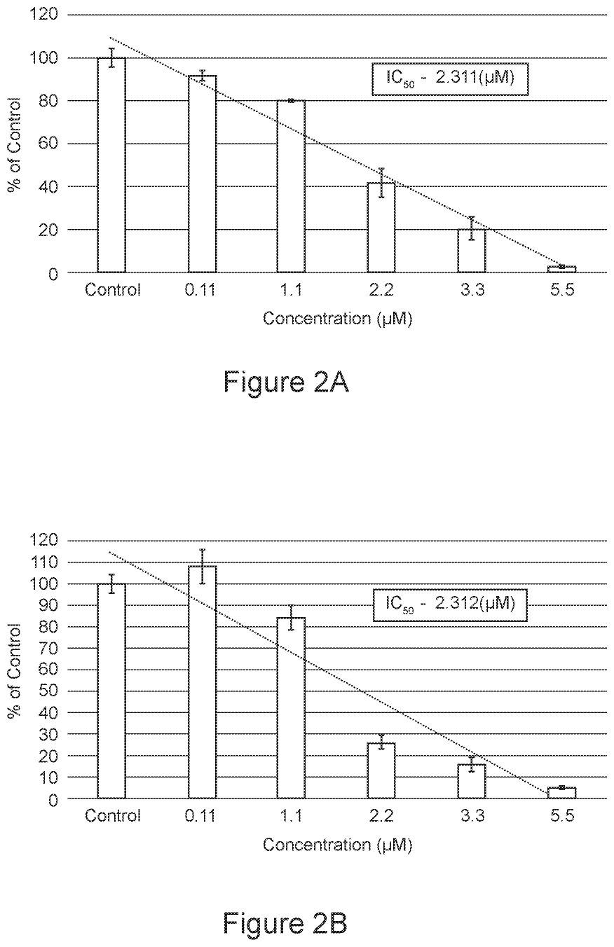

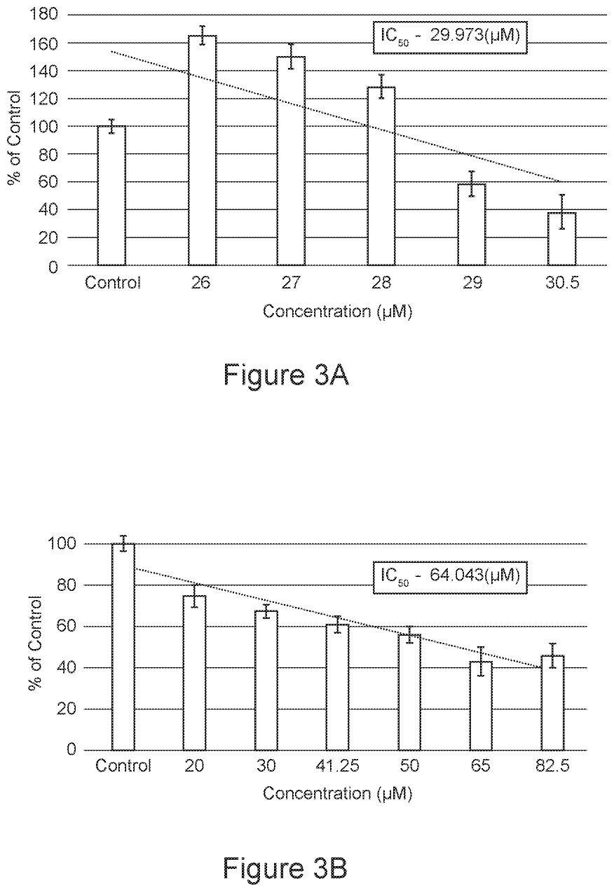

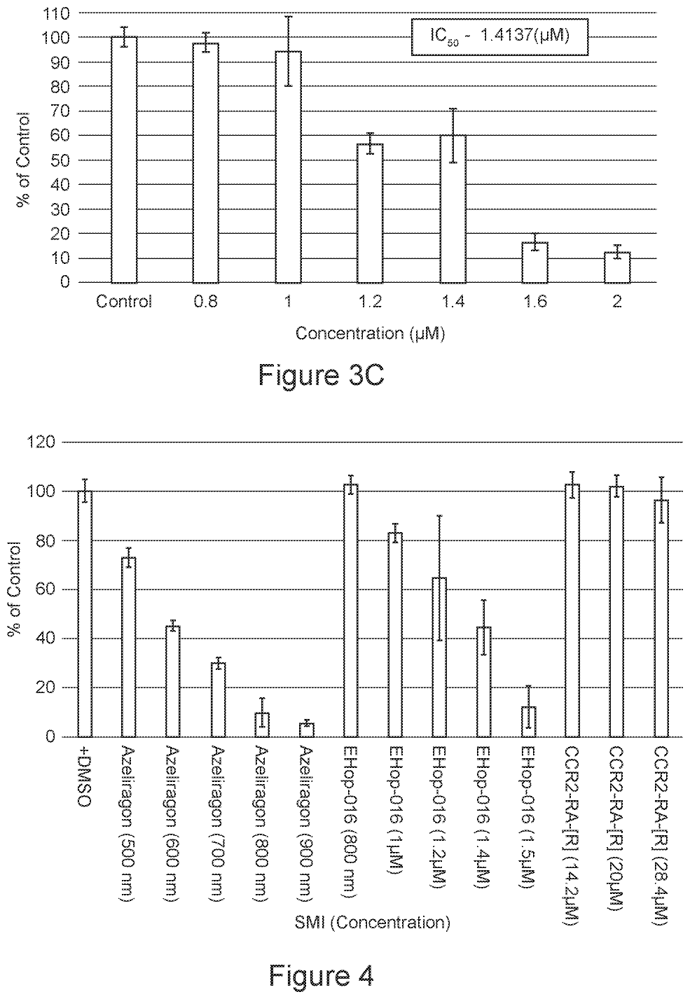

The effect of single 72-hour treatment with SMI on proliferation of HUVECs was examined and the inhibitory concentrations of 50% cell growth compared to control (IC₅₀) were calculated (FIGS. 2 and 3).

EHop-016 (FIG. 2A-FIG. 2B), 4′-Methoxyresveratrol (FIG. 3A), FPZ-ZM1 (FIG. 3B), and Azeliragon (FIG. 3C) showed linear inhibition of proliferation with increasing SMI concentration. Furthermore, the inhibition with EHop-016 was independent from the initial number of cells (FIG. 2A-FIG. 2B). The IC₅₀ values obtained in these experiments are summarized in Table 2.

TABLE 2
IC₅₀ concentrations of active and inactive SMIs

| Inhibitor | IC₅₀ |
|---|---|
| EHop-016 | 2.311 μM |
| Z62954982 | No effect |
| NSC 23766 trihydrochloride | No effect |
| 4′-Methoxyresveratrol | 64.043 μM |
| Azeliragon | 1.4137 μM |
| FPS-ZM1 | 29.973 μM |

Regarding the inhibitors of MCP-1/CCR2, the three SMIs caused a partial inhibition of up to 30% in HUVECs proliferation compared to control using their maximal attainable concentrations (Table 3). Hence, while it was impossible to determine the IC₅₀, proof of inhibition of HUVECs proliferation was provided.

TABLE 3
SMIs of MCP-1/CCR2 protein and inhibitory activity
of their maximal attainable concentrations.

| Inhibitor | Concentration | % of Inhibition ± SD |
|---|---|---|
| CCR2-RA-[R] | 28.421 μM | 33.4 ± 4.30 |
| PF-4136309 | 17.85 μM | 25.7 ± 7.68 |
| CCR2 antagonist | 20.99 μM | 13.8 ± 8.07 |
| 4 hydrochloride | | |

### Example 3: Optimizing the SMIs Effect by Longer Treatment Period

In order to enhance the inhibitory effect of the inactive SMI candidates (i.e., Z62954982 and NSC 23766 trihydrochloride) and of the SMIs that showed only partial effect (i.e., CCR2-RA-[R], PF-4136309, and CCR2 antagonist 4 hydrochloride), the treatment time was extended to 120 hours. Longer incubation time did not improve the inhibitory effect of in-active SMIs compared to 72-hour incubation.

### Example 4: Optimizing the SMIs Effect by Different Dosing Regimens

In order to enhance the inhibitory effect of the most active SMIs, different dosing regimens, including single dose (SD), alternate day dosing (ADD) and daily-dosing (DD) were examined for 72- and 120-hour treatment. Daily dosing (i.e., the medium was replaced daily with fresh SMI or a combination of SMIs three times for 72 hours starting 24 hours following cells seeding) decreased the IC₅₀ of both RAGE protein inhibitor, Azeliragon, and Rac1 protein inhibitor, EHop-016, by more than a half in the comparison with IC₅₀ of SD regimen when treated for 72 hours (FIG. 4, Table 4). The ability to lower the dosages while retaining the same inhibitory effect on proliferation of HUVECs allows to reduce the drug side effects resulting from toxicity and to obtain an isolated and specific effect of the SMIs.

TABLE 4
IC₅₀ of SMIs following daily dosing regimen.

| Inhibitor | IC₅₀ |
|---|---|
| EHop-016 | 1.239 μM |
| Azeliragon | 0.598 μM |

### Example 5: Examining the Inhibitory Effect of SMIs Combination Therapy on HUVECs Proliferation

Based on the previous results, the most effective SMIs (i.e., ones with the lowest IC₅₀) were selected for each of the target proteins for further testing the effect of combination therapy on proliferation of HUVECs. EHop-016 was selected for Rac1, Azeliragon for RAGE and CCR2-RA-[R] for MCP-1/CCR2. Proliferation inhibition test was performed for the different combinations of SMIs. The test was performed for three different periods of treatment (24, 48 and 72 hours). The combination of Azeliragon and EHop-016 (in the concentration of 1.2 and 1.3 μM, respectively) was the most effective in 48-hour and 72-hour treatments, while the addition of CCR2-RA-[R] (in the concentration of 22.74 μM) significantly augmented the proliferation inhibition effect in 24-hour treatment but not for 48- and 72-hour treatment (FIG. 5). Of note, augmented response with longer treatment time was observed for all SMI combinations.

### Example 6: Synergism Analysis of SMIs Combinations

Analysis of combined effect of the SMIs was determined from HUVECs proliferation results. SMIs synergy was determined by three synergy models.

FIGS. 6A, 6B and 6C show synergy analysis for combinations of Azeliragon and EHop-016 on HUVECs proliferation using ZIP model, Loewe model and HAS model, respectively. As can be seen from these figures, the combination of Azeliragon and EHop-016 showed synergistic activity in all tested concentrations.

### Example 7: The Effect of SMIs Combination on Downstream Effectors

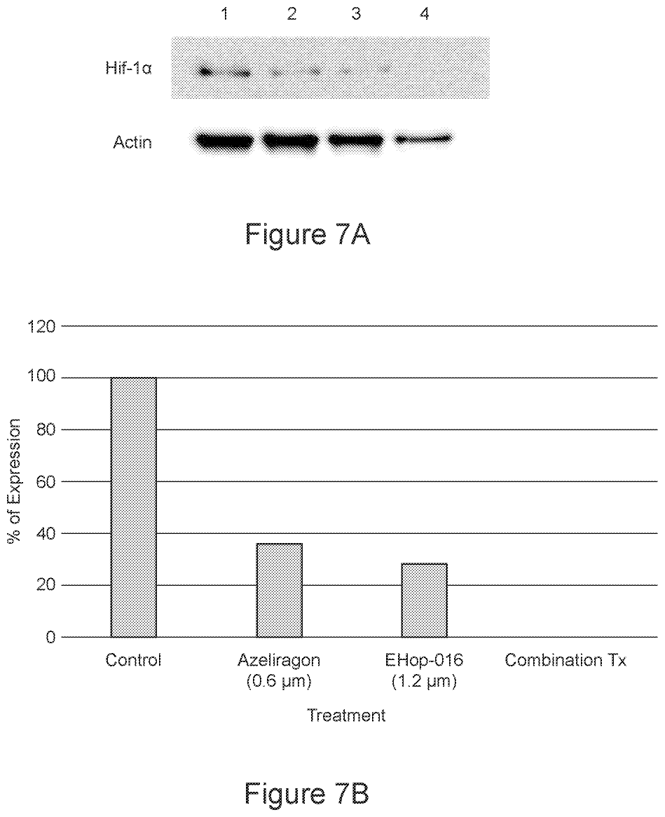

As can be seen in FIG. 7A, while each one of Azeliragon and EHop-016 inhibited expression of Hypoxia-inducible factor 1-alpha (HIF-1α), the combination of Azeliragon and EHop-016 (in the concentration of 0.6 μM and 1.3 μM, respectively) completely inhibited HIF-1α expression. FIG. 7B shows densitometry analysis of the results of FIG. 7A. Clearly, the combination of Azeliragon and EHop-016 inhibited significantly the expression of HIF-1α, a key protein involved in choroid neovascularization formation in wet AMD.

### Example 8: Examining the Apoptotic/Necrotic Side Effect of SMIs

In order to rule out toxic effects of the SMIs, Annexin V/propidium iodide (PI) assay was performed to evaluate apoptosis and necrosis in HUVECs following treatment with different concentrations of the selected SMIs. Annexin V detects cellular apoptosis, while PI detects necrotic or late apoptotic cells. It was found that the inhibitory effect of SMIs on proliferation observed in WST-1 test with the IC₅₀ of each SMI is way beyond the apoptotic/necrotic side effect of SMIs (Data not shown).

### Example 9: The Effect of Selected SMIs on the Migration of HUVECs

Endothelial cells migration is considered another fundamental process in angiogenesis. Hence, the effect of the selected SMIs (i.e., the most effective proliferation inhibitors) on the migration of HUVECs was examined in vitro, using a scratch test. A live cell imaging system (Incucyte®) was used to obtain serial photos of the migrating cells.

It was found that the Rac1 inhibitor, Ehop-016, is the strongest migration inhibitor (FIG. 8A-FIG. 8C). Despite the fact that CCR2-RA-[R], did not show significant inhibition of HUVECs proliferation, it strongly inhibited cell migration (FIG. 9C). On the other hand, Azeliragon aimed against RAGE protein and the most potent proliferation inhibitor, showed only modest migratory inhibition compared to Ehop-016 and CCR2-RA-[R] (data not shown). The efficacy of SMIs in proliferation and migration assays is summarized in Table 4.

TABLE 4
Summary of the SMIs effect on proliferation
and migration of HUVECs

| | | Proliferation | Migration |
|---|---|---|---|
| Function | SMI | assay | assay |
| Proangiogenic | Azeliragon | ++++ | + |
| Oxidative Stress | Ehop-016 | +++ | +++ |
| Proinflammatory | CCR2-RA-[R] | + | +++ |

### Example 10: Examining the Specificity of the SMIs Effect on HUVECs Compared to Other Cell Lines

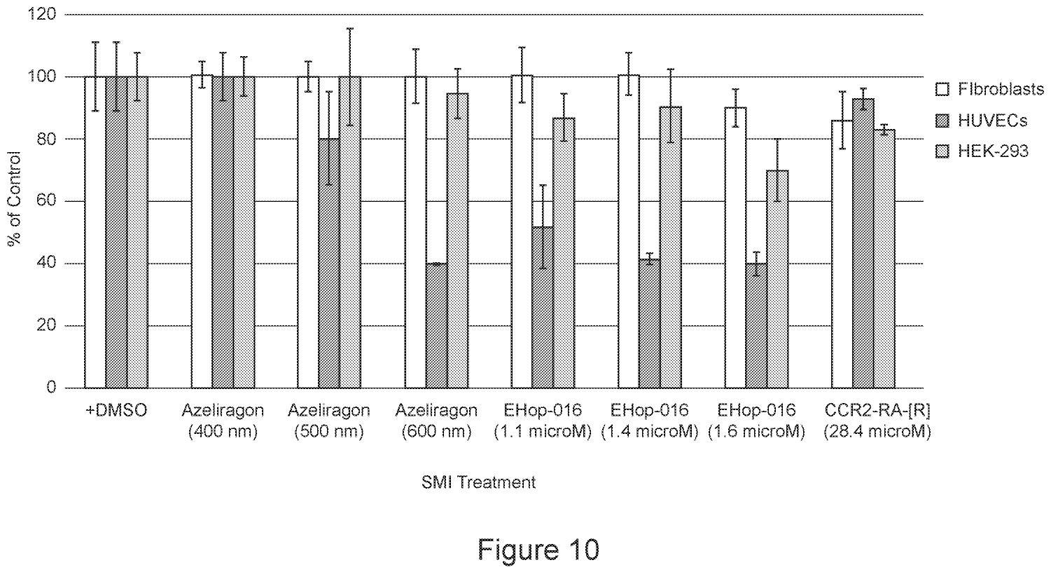

In order to determine whether the inhibitory effect of the SMIs is specific to endothelial cells (EC) which expresses high levels of RAGE, Rac1 and MCP-1/CCR2, the effect of SMIs on proliferation of fibroblasts and human embryonic kidney 293 (HEK-293) cell lines was compared to HUVECs using WST-1 test. The inhibition test with daily dosing with the IC₅₀ of the selected SMIs for 72-hours showed that the inhibition of proliferation is specific to HUVECs while not affecting HEK-293 and fibroblasts at the used concentrations (FIG. 10).

Based on on-line information, https://www.proteinatlas.org, it was found that the proteins Rac1, RAGE and MCP-1/CCR2 were found to be highly expressed in EC compared to other cell types of the retina.

### Example 11: Choroid Sprouting Assay to Assess the Effect of SMIs on Angiogenesis, Ex-Vivo

The anti-angiogenic effect of SMIs was studied in ex-vivo choroid sprouting model. The assay results are presented on (FIG. 11A-FIG. 11F). Clearly, both Azeliragon (FIG. 11C) and Ethop-016 (FIG. 11D) inhibited the area of the sprouting vessels compared to untreated cells (FIG. 11A) and cells treated with dimethyl sulfoxide (DMSO, solvent, FIG. 11B). Moreover, combination of both SMIs inhibited even more the neovascularization of the choroid explant compared to each one of the SMIs alone, indicating the augmented efficacy of the combination therapy (FIG. 11E-FIG. 11F).

### Example 12: Laser-Induced Choroidal Neovascularization (CNV) Model—In Vivo Studies

The therapeutic effect of the SMIs combinations Azeliragon and EHop-016 was tested in Laser-Induced CNV model, a model for wet AMD. As can be seen from FIGS. 12A and 12B, Isolectin staining of CNV lesions was increased in sham mice (FIG. 12B) as compared with treated mice (FIG. 12A). These results demonstrate that the combination treatment of Rac1 and RAGE SMIs was effective in inhibiting formation of choroidal neovascularization. The results are in accordance with the in vitro and ex vivo data showing that the high efficacy of the combination formula in inhibiting angiogenesis.

As can be seem from the histogram in FIG. 13, the CNV area, stained with Isolectin and analyzed using ImageJ software, was statistically significant lower in the mice treated with intravitreal injections of Rac1 and RAGE SMIs combination formula compared to mice treated with sham.

Further, as shown in FIG. 14, the CNV area measured for the mice treated with Ehop-016, Rac1 inhibitor, as part of the dosage calibration assays in vivo, was comparable to the dramatic effect received from Aflibercept injection in Laser-induced CNV model.

### Example 13: In Vivo Studies

The therapeutic effect of the SMIs combinations is tested in various neovascularization and non-neovascularization mouse models including laser-induced CNV (a model for nAMD), STZ-Induced retinopathy (a model for diabetic retinopathy), Oxygen induced retinopathy (a model for retinopathy of prematurity), Light-induced retinal degeneration (a model for dry AMD), Experimental autoimmune uveitis (EAU) (a model for uveitis), Intravitreal dispose injection (a model for proliferative vitreoretinopathy (PVR)), Retinal degeneration 10 (rd10) (inherited retinal degeneration (IRD) model) and many other retinal disease models.

For the neovascularization mouse models, the ability of the SMIs combination treatment to regress, inhibit and prevent the development of abnormal retinal and choroidal vessels together with the associated functional and anatomical damages is examined using fundoscopy, fundus color and autofluorescence photos, optical coherence tomography (OCT) and fluorescein angiography (FA), immunohistochemistry and immunofluorescence staining of retinal sections and choroidal-RPE flat-mount specimens. The visual function is tested for visual acuity and full-field electroretinogram (ffERG) performed to rule out retinal toxicity of the SMIs and combinations thereof.

For the non-neovascularization mouse models, the ability of the SMIs combination treatment to inhibit and prevent both functional and anatomical retinal damages caused mainly by inflammatory response (in uveitis model), retinal atrophy and degeneration (in dry AMD and inherited retinal degeneration models), retinal fibrosis (proliferative vitreoretinopathy (PVR) model). This is performed using fundoscopy, fundus color and autofluorescence photos, optical coherence tomography (OCT) and fluorescein angiography (FA), immunohistochemistry and immunofluorescence stains of retinal sections and choroidal-RPE flat-mount specimens. The visual function is tested for visual acuity and full-field electroretinogram (ffERG) performed to rule out retinal toxicity of the developed SMIs.

### Example 14: Development of Drug Formulation

The SMIs drug combination are formulated for convenient, extended and optimal drug delivery into the eye. Nano-based drug delivery systems are used in this development process. The first set of formulations is based on nano emulsions using biodegradable polymers e.g., poly-lactic-co-glycolic acid (PLGA) with pre-designated degradation mechanisms.

1. A combination for treating a retinal disease, the combination comprising at least two inhibitors of respectively at least two proteins of different signaling pathways of retinal disease.

2. The combination of claim 1, wherein said at least two proteins are selected from the group consisting of CCR2, RAGE and Rac-1.

3. The combination of claim 1, wherein the at least two proteins are selected from the group consisting of MCP-1, RAGE and Rac-1.

4. The combination of any one of claims 1 to 3, wherein said at least two inhibitors are at least two small molecule inhibitors (SMIs).

5. The combination of claim 4, wherein each of said at least two SMIs fulfills one or more of the following criteria:
it has a molecular weight of less than 600 Da;
it has no more than 5 hydrogen bond donors;
it has no more than 10 hydrogen bond acceptors; and
it has a partition coefficient not greater than 5.

6. The combination of claim 4 or 5, wherein said at least two SMIs, at least one SMI inhibits at least the RAGE protein and at least one other SMI inhibits at least the Rac1 protein.

7. The combination of claim 4 or 5, wherein said at least two SMIs, at least one SMI inhibits at least the RAGE protein and at least one other SMI inhibits at least the MCP-1 protein.

8. The combination of claim 4 or 5, wherein said at least two SMIs, at least one SMI inhibits at least the RAGE protein and at least one other SMI inhibits at least the CCR2 protein.

9. The combination of claim 4 or 5, wherein said at least two SMIs, at least one SMI inhibits at least the Rac1 protein and at least one other SMI inhibits at least the MCP-1 protein.

10. The combination of claim 4 or 5, wherein said at least two SMIs, at least one SMI inhibits at least the Rac1 protein and at least one other SMI inhibits at least the CCR2 protein.

11. The combination of any one of claims 4 to 10, wherein said SMI that inhibits at least the Rac1 protein has a chemical structure of one or more of Formula I, IV, V, VI, VII or a solvate, a hydrate, a stereoisomer, a pharmaceutically acceptable prodrug, a pharmaceutically active metabolite, a pharmaceutically acceptable salt, a crystalline form, an amorphous form, a physiologically functional analogue, a physiologically functional derivative thereof or a combination thereof.

12. The combination of claim 11, wherein said SMI has a chemical structure of Formula I:

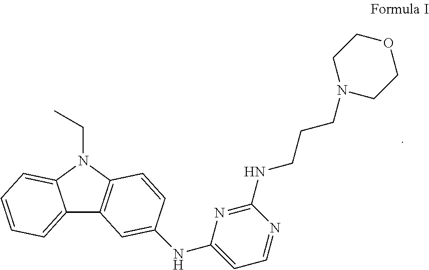 [SMILES: CCn1c2ccccc2c2cc(Nc3ccnc(NCCCN4CCOCC4)n3)ccc21]

13. The combination of claim 11, wherein said SMI is a functional analog of Formula I.

14. The combination of any one of claims 4 to 10, wherein said SMI that inhibits at least the RAGE protein has a chemical structure of one or more of Formula II, VIII, IX or a solvate, a hydrate, a stereoisomer, a pharmaceutically acceptable prodrug, a pharmaceutically active metabolite, a pharmaceutically acceptable salt, a crystalline form, an amorphous form, a physiologically functional analogue, a physiologically functional derivative thereof or a combination thereof.

15. The combination of claim 14, wherein said SMI has a chemical structure of Formula II:

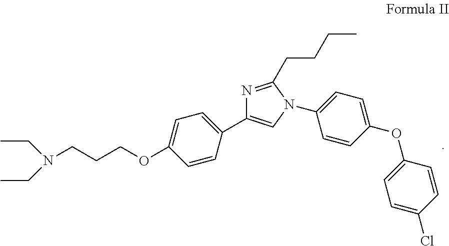 [SMILES: CCCCc1nc(-c2ccc(OCCCN(CC)CC)cc2)cn1-c1ccc(Oc2ccc(Cl)cc2)cc1]

16. The combination of claim 14, wherein said SMI is a functional analog of Formula II.

17. The combination of any one of claims 4 to 10, wherein said SMI that inhibits at least the CCR2 protein has a chemical structure of one or more of Formula III, XI, XII or a solvate, a hydrate, a stereoisomer, a pharmaceutically acceptable prodrug, a pharmaceutically active metabolite, a pharmaceutically acceptable salt, a crystalline form, an amorphous form, a physiologically functional analogue, a physiologically functional derivative thereof or a combination thereof.

18. The combination of claim 17, wherein said SMI has having a chemical structure of Formula III:

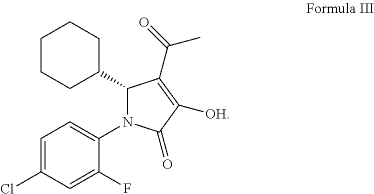 [SMILES: CC(=O)C1=C(O)C(=O)N(c2ccc(Cl)cc2F)[C@@H]1C1CCCCC1]

19. The combination of claim 17, wherein said SMI is a functional analog of Formula III.

20. The combination of any one of claims 4 to 19, wherein said at least two SMIs are selected from (i) at least one SMI having Formula (I), (IV), (V), (VI) or (VII); (ii) at least one SMI having Formula (II), (VIII), (IX) or (X); (iii) at least one SMI having Formula (III), (XI) or (XII) or a solvate, a hydrate, a stereoisomer, a pharmaceutically acceptable prodrug, a pharmaceutically active metabolite, a pharmaceutically acceptable salt, a crystalline form, an amorphous form, a physiologically functional analogue, a physiologically functional derivative thereof or a combination thereof.

21. The combination of any one of claims 4 to 20, wherein said at least two SMIs are selected from (i) at least one SMI having Formula (I), (IV) or (V); (ii) at least one SMI having Formula (II), (VIII), (IX) or (X); (iii) at least one SMI having Formula (III), (XI) or (XII) or a solvate, a hydrate, a stereoisomer, a pharmaceutically acceptable prodrug, a pharmaceutically active metabolite, a pharmaceutically acceptable salt, a crystalline form, an amorphous form, a physiologically functional analogue, a physiologically functional derivative thereof or a combination thereof.

22. The combination of any one of claims 1 to 21, wherein said retinal disease is selected from the group consisting of wet (neovascular) age-related macular degeneration (nAMD), diabetic retinopathy (DR, including proliferative diabetic retinopathy (PDR), and non-proliferative diabetic retinopathy (nPDR)), diabetic macular oedema (DMO), retinopathy of prematurity, radiation retinopathy, hypertensive retinopathy, myopic choroidal neovascularization (CNV), retinal vein occlusion (RVO), retinal artery occlusion (RAO), vasoproliferative tumor, coat's diseases, familial exudative vitreoretinopathy (FEVR), uveitis induced CNV, inherited retinal degeneration associated CNV, sickle cell retinopathy, idiopathic choroidal neovascularization, Irvine-Gass syndrome, choroidal hemangioma, dry age-related macular degeneration (AMD), retinal atrophy, carcinoma associated retinopathy (CAR), autoimmune induced retinopathy (AIR), central serous chorioretinopathy (CSCR), uveitis, inherited retinal degeneration (IRD), cystoid macular edema, proliferative vitreoretinopathy (PVR), Polypoidal choroidal vasculopathy (PCV).

23. The combination of any one of claims 1 to 22, wherein said retinal disease is a retinal vascular disease.

24. The combination of claim 23, wherein said retinal vascular disease is selected from the group consisting of wet (neovascular) age-related macular degeneration (nAMD), diabetic retinopathy (DR), proliferative diabetic retinopathy (PDR) and diabetic macular oedema (DMO), and retinal vein occlusion (RVO).

25. The combination of any one of claims 4 to 24, wherein at least one of the at least two SMIs is formulated within a delivery system.

26. The combination of claim 25, wherein said delivery system is for controlled delivery of the SMI.

27. The combination of any one of claims 4 to 24, wherein said at least two SMIs are formulated for sequential or concomitant administration.

28. The combination of any one of claims 4 to 24, wherein said at least two SMIs are formulated in the same or different composition.

29. A pharmaceutical composition comprising a pharmaceutically acceptable carrier and at least two SMIsof respectively at least two proteins of different signaling pathways associated with at least a retinal disease.

30. The pharmaceutical composition of claim 20, wherein said at least two SMIs are as defined in any one of claims 4 to 21.

31. The pharmaceutical composition of claim 29 or 30, formulated for topical administration.

32. The pharmaceutical composition of any one of claims 29 to 31, wherein at least one of the at least two SMIs is formulated within a delivery system.

33. The pharmaceutical composition of claim 32, wherein said delivery system is for controlled delivery of the SMI.

34. The pharmaceutical composition of any one of claims 29 to 33, wherein the pharmaceutically acceptable carrier is selected from the group comprising polylactic acid (PLA), poly-lactic-co-glycolic acid (PLGA), polyvinyl alcohol (PVA), polyethyleneimine (PEI) and combinations thereof.

35. The pharmaceutical composition of any one of claims 29 to 34, wherein said retinal disease is selected from the group consisting of nAMD, DR, PDR, nPDR, DMO, retinopathy of prematurity, radiation retinopathy, hypertensive retinopathy, CNV, RVO, RAO), vasoproliferative tumor, coat's diseases, FEVR, uveitis induced CNV, inherited retinal degeneration associated CNV, sickle cell retinopathy, idiopathic choroidal neovascularization, Irvine-Gass syndrome, choroidal hemangioma, AMD, retinal atrophy, CAR, AIR, CSCR, IRD, cystoid macular edema, PVR, PCV.

36. The pharmaceutical composition of any one of claims 29 to 34, wherein said retinal disease is a retinal vascular disease.

37. The pharmaceutical composition of claim 36, wherein said retinal vascular disease is selected from the group consisting of wnAMD, DR, PDR, DMO, and RVO.

38. A method of treating a retinal disease, the method comprises administering to a subject in need of said treatment at least two SMIs of respectively at least two proteins of different signaling pathways of retinal disease.

39. The method of claim 38, wherein said at least two SMIs are as defined in any one of claims 4 to 21.

40. The method of claim 38 or 39, comprising administering at least one of said at least two SMIs topically.

41. The method of any one of claims 38 to 40, comprising administering at least one of said at least two SMIs by injection.

42. The method of any one of claims 38 to 41, wherein at least one of the at least two SMIs is formulated within a delivery system.

43. The method of claim 42, wherein said delivery system is for controlled delivery of the SMI.

44. The method of any one of claims 38 to 43, comprising sequential or concomitant administration of the at least two SMIs.

45. The method of any one of claims 38 to 43, comprising administration of the at least two SMIs in the same composition.

46. The method of any one of claims 38 to 45, wherein said retinal disease is selected from the group consisting of nAMD, DR, PDR, nPDR, DMO, retinopathy of prematurity, radiation retinopathy, hypertensive retinopathy, CNV, RVO, RAO), vasoproliferative tumor, coat's diseases, FEVR, uveitis induced CNV, inherited retinal degeneration associated CNV, sickle cell retinopathy, idiopathic choroidal neovascularization, Irvine-Gass syndrome, choroidal hemangioma, AMD, retinal atrophy, CAR, AIR, CSCR, IRD, cystoid macular edema, PVR, PCV.

47. The method of any one of claims 38 to 45, wherein said retinal disease is a retinal vascular disease.

48. The method of claim 47, wherein said retinal vascular disease is selected from the group consisting of nAMD, DR, PDR, DMO, and RVO.

49. A package or kit comprising
a first SMI targeted against a protein of a signaling pathway of a retinal disease;
at least one additional SMI being different from said first SMI and targeted at a different protein of a signaling pathway of said retinal disease; and
instructions for use of a combination of said first SMI and said at least one additional SMI for treating said retinal disease.

50. The package or kit of claim 49, wherein said first SMI is separated from said at least one additional SMI.
